(19) United States
(12) Patent Application Publication
(10) Pub. No.: US 2021/0299187 A1
(43) Pub. Date: Sep. 30, 2021

(54) CANCER THERAPY

(71) Applicant: IMMODULON THERAPEUTICS LIMITED, LONDON (GB)

(72) Inventors: GLEN MARTYN, UXBRIDGE (GB); JAKOB KAMPINGA, UXBRIDGE (GB); GRAHAM BURTON, UXBRIDGE (GB); ANGUS DALGLEISH, UXBRIDGE (GB)

(21) Appl. No.: 17/255,447

(22) PCT Filed: Jun. 25, 2019
(86) PCT No.: PCT/GB2019/051794

§ 371 Date: Dec. 23, 2020

(30) Foreign Application Priority Data
Jun. 25, 2018 (EP) 18179675.6

(51) Int. Cl. A61K 35/74; A61K 39/395; A61K 45/06; A61K 9/00; C07K 16/28; A61P 35/00; A61M 37/00

(52) U.S. Cl. CPC A61K 35/74; A61K 39/3955; A61K 45/06; A61K 9/0021; A61K 2039/505; A61P 35/00; C07K 16/2818; A61M 37/0015; C07K 16/2827

(57) ABSTRACT

A non-viable, whole-cell Mycobacterium for use in the treatment, reduction, inhibition or control of one or more tumours in a checkpoint inhibitor refractory patient, wherein said checkpoint inhibitor refractory patient is intended to undergo checkpoint inhibition therapy simultaneously, separately or sequentially with administration of the Mycobacterium, and/or co-stimulatory checkpoint therapy, and optionally further comprising administering one or more additional anticancer treatments or agents.

### FIELD OF THE INVENTION

The present invention relates to the field of cancer therapy. In particular, the present invention relates to a method of preventing, treating or inhibiting the development of tumours and/or metastases in a subject who is refractory (resistant) to checkpoint inhibitor therapy.

### BACKGROUND OF THE INVENTION

In humans with advanced cancer, anti-tumour immunity is often ineffective due to the tightly regulated interplay of pro- and anti-inflammatory, immune-stimulatory and immunosuppressive signals. It is now believed that the immune system constantly monitors and eliminates newly transformed cells. Accordingly, cancer cells may alter their phenotype in response to immune pressure in order to escape attack (immunoediting) and upregulate expression of inhibitory signals. Through immunoediting and other subversive processes, primary tumour and metastasis maintain their own survival. One of the major mechanisms of anti-tumour immunity subversion is known as ‘T-cell exhaustion’, which results from chronic exposure to antigens and is characterized by the up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (TIM-3), Lymphocyte Activation Gene-3 (LAG-3; CD223), and others are often referred to as a checkpoint regulators. They act as molecular “tollbooths,” which allow extracellular information to dictate whether cell cycle progression and other intracellular signalling processes should proceed.

In addition to specific antigen recognition through the TCR, T-cell activation is regulated through a balance of positive and negative signals provided by co-stimulatory receptors. These surface proteins are typically members of either the TNF receptor or B7 superfamilies. Agonistic antibodies directed against activating co-stimulatory molecules and blocking antibodies against negative co-stimulatory molecules may enhance T-cell stimulation to promote tumour destruction.

Programmed Cell Death Protein 1, (PD-1 or CD279), a 55-kD type 1 transmembrane protein, is a member of the CD28 family of T cell co-stimulatory receptors that include immunoglobulin superfamily member CD28, CTLA-4, inducible co-stimulator (ICOS), and BTLA. PD-1 is highly expressed on activated T cells and B cells. PD-1 expression can also be detected on memory T-cell subsets with variable levels of expression. Two ligands specific for PD-1 have been identified: programmed death-ligand 1 (PD-L1, also known as B7-H1 or CD274) and PD-L2 (also known as B7-DC or CD273). PD-L1 and PD-L2 have been shown to down-regulate T cell activation upon binding to PD-1 in both mouse and human systems (Okazaki et al., Int Immunol., 2007; 19: 813-824). The interaction of PD-1 with its ligands, PD-L1 and PD-L2, which are expressed on antigen-presenting cells (APCs) and dendritic cells (DCs), transmits negative regulatory stimuli to down-modulate the activated T cell immune response. Blockade of PD-1 suppresses this negative signal and amplifies T cell responses.

Numerous studies indicate that the cancer microenvironment manipulates the PD-L1-/PD-1 signalling pathway and that induction of PD-L1 expression is associated with inhibition of immune responses against cancer, thus permitting cancer progression and metastasis. The PD-L1/PD-1 signalling pathway is a primary mechanism of cancer immune evasion for several reasons. First, and most importantly, this pathway is involved in negative regulation of immune responses of activated T effector cells, found in the periphery. Second, PD-L1 is up-regulated in cancer microenvironments, while PD-1 is also up-regulated on activated tumour infiltrating T cells, thus possibly potentiating a vicious cycle of inhibition. Third, this pathway is intricately involved in both innate and adaptive immune regulation through bi-directional signalling. These factors make the PD-1/PD-L1 complex a central point through which cancer can manipulate immune responses and promote its own progression.

The first immune-checkpoint inhibitor to be tested in a clinical trial was ipilimumab (Yervoy, Bristol-Myers Squibb), a CTLA-4 mAb. Anti-CTLA-4 mAb is a powerful checkpoint inhibitor which removes “the break” from both naïve and antigen-experienced cells. Therapy enhances the antitumor function of CD8+ T cells, increases the ratio of CD8+ T cells to Foxp3+T regulatory cells, and inhibits the suppressive function of T regulatory cells. The major drawback to anti-CTLA-4 mAb therapy is the generation of autoimmune toxicities.

TIM-3 has been identified as another important inhibitory receptor expressed by exhausted CD8+ T cells. In mouse models of cancer, it has been shown that the most dysfunctional tumour-infiltrating CD8+ T cells actually co-express PD-1 and TIM-3.

LAG-3 is another recently identified inhibitory receptor that acts to limit effector T-cell function and augment the suppressive activity of T regulatory cells. It has recently been revealed that PD-1 and LAG-3 are extensively co-expressed by tumour-infiltrating T cells in mice, and that combined blockade of PD-1 and LAG-3 provokes potent synergistic antitumor immune responses in mouse models of cancer.

Currently, antagonist mAbs against both PD-1 and their ligand PD-L1 are in various stages of development for the treatment of cancer, and recent human trials have shown promising results in cancer patients with advanced disease.

The first of the agents blocking the B7-H1/PD-1 pathway to enter phase I clinical trials was Nivolumab (MDX-1106/BMS-936558/ONO-4538), a fully human IgG4 anti-PD-1 mAb developed by Bristol-Myers Squibb.

The results of a phase II study that compared combined nivolumab and ipilimumab with ipilimumab alone in patients with BRAF wild-type melanoma showed objective response rates of 61% with the combination therapy and 11% with the monotherapy, with complete responses in 22% and 0% of patients, respectively. Treatment-related adverse events of grade 3 or 4 were reported in 54% of the patients in the combination group and in 24% of those in the ipilimumab group. Treatment-related adverse events of any grade that led to discontinuation of the study drug occurred in 7.7% of the patients in the nivolumab group, 36.4% of those in the nivolumab-plus-ipilimumab group, and 14.8% of those in the ipilimumab group.

Immune checkpoint inhibitor therapy has been particularly successful in melanoma, for which approved treatments now include anti-PD-1 (nivolumab and pembrolizumab), anti-CTLA-4 (ipilimumab), and combination anti-PD-1/CTLA-4 regimens (nivolumab-ipilimumab). Long-term survival data for patients with melanoma treated with ipilimumab (antiCTLA-4) indicates 20% of patients show evidence of continued durable disease control or response 5-10 years after starting therapy. The response rate for melanoma patients treated with pembrolizumab (anti-PD-1) was 33% at 3 years with 70-80% of patients initially responding maintaining clinical response.

A phase III study showed an increase in the median PFS of patients treated with nivolumab and ipilimumab (11.5 months; HR, 0.42, P<0.001) and nivolumab alone (6.9 months; HR, 0.57, P<0.001) compared with ipilimumab alone (2.9 months). At a minimum follow-up of 28 months, the median OS had not been reached in the combination or nivolumab-alone groups and was 20 months for ipilimumab alone [HR: combination vs. ipilimumab, 0.55 (P<0.0001); nivolumab vs. ipilimumab, 0.63 (P<0.0001); ref. 16]. The two-year OS rates were 64%, 59%, and 45% in the combination, nivolumab, and ipilimumab groups, respectively.

The results of these clinical trials highlight the significant impact immunotherapies have had on the clinical management of patients with advanced-stage metastatic melanoma. However, although approximately 35% to 60% of patients have a RECIST (response evaluation criteria in solid tumours) response (10%-12% a complete response) to anti-PD-1-based immunotherapy, 40% to 65% have shown minimal or no RECIST response at the outset, and 43% of responders develop acquired resistance by 3 years.

Analysis of clinical trial data can identify three broad populations of patients—(1) those that respond initially and continue to respond (responders), (2) those that fail to ever respond (innate resistance), and (3) those that initially respond but eventually develop disease progression (acquired resistance).

Therefore, despite the unprecedented durable response rates observed with cancer immunotherapies, the majority of patients do not benefit from the treatment (primary resistance), and some responders relapse after a period of response (acquired resistance). Several common cancer types have shown very low frequency of response (breast, prostate, and colon cancers), and heterogeneous responses have been seen even between distinct tumours within the same patient. Mechanisms of innate and acquired resistance to checkpoint inhibitor therapy are not fully understood, owing in part to the incomplete understanding of the full complement of clinical, molecular, and immunologic factors associated with clinical response and long-term benefit to checkpoint inhibitor therapy. In addition, few immune competent pre-clinical models exist in which tumour regression is induced by checkpoint inhibitors, limiting the ability to reproduce the diversity of tumour-immune interactions in patients.

Patients who have primary resistance to checkpoint inhibitors do not respond to the initial therapy. Ongoing studies indicate that both tumour-cell-intrinsic and tumour-cell-extrinsic factors contribute to the resistance mechanisms.

Factors that lead to primary or adaptive resistance include: lack of antigenic mutations, T-cell exhaustion, lack of sufficient or suitable tumour antigen presentation and/or processing, impaired DC maturation, loss of HLA expression, alterations of several signalling pathways (MAPK, PI3K, JAK. STAT, WNT, IFN), induction of IDO, upregulation of CD73, constitutive PD-L1 expression, impaired intratumoral immune cell infiltration (e.g. T cells), activation of alternate immune inhibitory checkpoints (e.g. VISTA, LAG, TIM-3), activation of metabolic/inflammatory mediators, overexpression of VEGF, and activation of immunosuppressive cells, e.g. tumour associated macrophages (TAMs), regulatory T-cells (Tregs), myeloid derived suppressive cells (MDSCs), etc.

Other factors that are associated with acquired resistance of cancer, include loss of target antigen, HLA, and altered interferon signalling, as well as loss of T cell functionality or epigenetic changes thereon.

Immune suppressive cell types that have been shown to influence checkpoint inhibitor efficacy in pre-clinical models include Tregs, MDSCs, Th2 CD4^{b} T cells, and M2-polarised tumour-associated macrophages. These cell types individually and collectively promote an immune suppressive tumour microenvironment (TME) that prevent anti-tumour cytotoxic and Th1-directed T-cell activities, primarily through the release of cytokines, chemokines, and other soluble mediators. Depletion of these immune suppressive cell types (e.g., MDSCs and Tregs) has experimentally been shown to enhance anti-tumour immune responses overcoming innate resistance.

A higher Treg:Teffector cell (Teff) ratio within tumour tissue is associated with worse prognoses in many cancers, including ovarian cancer, pancreatic ductal adenocarcinoma, lung cancer, glioblastoma, non-Hodgkin's lymphoma, melanoma and other malignancies. Accordingly, tumours for which a therapy is unable to increase Teffs and/or deplete Tregs to increase the ratio of Teffs to Tregs, are likely to be resistant to treatment, either initially or during the relapsed disease setting.

Accordingly, an aim of the present invention is to provide a combination therapy for treating cancer in patients identified as resistant to checkpoint inhibitor therapy. This combination therapy comprises non-viable whole-cell Mycobacterium and blockade of checkpoint inhibitors, wherein the therapy has the potential to overcome such innate or acquired resistance to checkpoint inhibitor therapy.

### SUMMARY OF THE INVENTION

The present invention provides an effective method for treating and/or preventing cancer and/or the establishment of metastases, in a checkpoint inhibitor refractory patient, by administering a checkpoint inhibitor which acts synergistically with a non-viable, whole cell Mycobacterium.

In a first aspect of the invention, there is provided a non-viable, whole-cell Mycobacterium for use in the treatment, reduction, inhibition or control of one or more tumours in a checkpoint inhibitor refractory patient, wherein said checkpoint inhibitor refractory patient is intended to undergo checkpoint inhibition therapy simultaneously, separately or sequentially with administration of the Mycobacterium.

In a second aspect of the invention, there is provided a non-viable, whole-cell Mycobacterium for use in the treatment, reduction, inhibition or control of one or more tumours in a checkpoint inhibitor refractory patient, wherein said checkpoint inhibitor refractory patient is intended to undergo checkpoint inhibition therapy simultaneously, separately or sequentially with administration of the Mycobacterium, wherein said checkpoint inhibition therapy comprises administration of one or more blocking agents, selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3 and combinations thereof.

In a third aspect of the invention, there is provided a non-viable, whole-cell Mycobacterium for use in the treatment, reduction, inhibition or control of one or more tumours in a checkpoint inhibitor refractory patient, wherein said checkpoint inhibitor refractory patient is intended to undergo checkpoint inhibition therapy simultaneously, separately or sequentially with administration of the Mycobacterium and further comprising co-stimulatory checkpoint therapy, simultaneously, separately or sequentially with administration of the Mycobacterium, wherein said co-stimulatory checkpoint therapy comprises administration of one or more binding agents selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CD27, CD28, CD40, CD122, CD137, OX40, GITR, ICOS and combinations thereof.

In a fourth aspect of the invention, there is provided a non-viable whole-cell Mycobacterium for use in the treatment, reduction, inhibition or control of one or more tumours in a checkpoint inhibitor refractory patient, wherein said checkpoint inhibitor refractory patient is intended to undergo checkpoint inhibition therapy simultaneously, separately or sequentially with administration of the Mycobacterium optionally further comprising co-stimulatory checkpoint therapy, and further comprising administering one or more additional anticancer treatments or agents, simultaneously, separately or sequentially with administration of the Mycobacterium, and/or checkpoint inhibition therapy and/or the co-stimulatory checkpoint therapy, wherein the one or more additional anticancer treatments or agents is selected from: adoptive cell therapy, surgical therapy, chemotherapy, radiation therapy, hormonal therapy, small molecule therapy such as metformin, receptor kinase inhibitor therapy, hyperthermia treatment, phototherapy, radioablation therapy, anti-angiogenic therapy, cytokine therapy, cryotherapy, biological therapy, HDAC inhibitor e.g. OKI-179, BRAF inhibitor, MEK inhibitor, EGFR inhibitor, VEGF inhibitor, PI3K delta inhibitor, PARP inhibitor, mTOR inhibitor, hypomethylating agents, oncolytic virus, TLR agonist including TLR2, 4, 7, 8 or 9 agonists, or TLR 5 agonists such as MRx0518 (4D Pharma), STING agonists (including MIW815 and SYNB1891), and cancer vaccines such as GVAX or CIMAvax.

In a fifth aspect of the invention, there is provided a method of treating, reducing, inhibiting or controlling a neoplasia, tumour or cancer in a checkpoint inhibitor refractory subject, wherein said method comprises simultaneously, separately or sequentially administering to the subject, (i) one or more checkpoint inhibitors, selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3 and combinations thereof, and (ii) a non-viable, whole cell Mycobacterium, wherein said method results in enhanced therapeutic efficacy relative to administration of the one or more checkpoint inhibitors or non-viable, whole cell Mycobacterium alone, and optionally wherein said method comprises administration of a sub-therapeutic amount of said checkpoint inhibitor.

In a sixth aspect of the invention, there is provided a method of treating, reducing, inhibiting or controlling a neoplasia, tumour or cancer in a checkpoint inhibitor refractory subject, wherein said method comprises simultaneously, separately or sequentially administering to the subject, (i) one or more checkpoint inhibitors, selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3 and combinations thereof, (ii) a non-viable, whole cell Mycobacterium, and (iii), co-stimulatory checkpoint therapy, wherein said co-stimulatory checkpoint therapy comprises administration of one or more binding agents, selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CD27, CD28, CD40, CD122, CD137, OX40, GITR, ICOS and combinations thereof, wherein said method results in enhanced therapeutic efficacy relative to administration of the one or more checkpoint inhibitors, co-stimulatory checkpoint therapy, or non-viable, whole cell Mycobacterium alone.

In a seventh aspect of the invention, there is provided a method of treating, reducing, inhibiting or controlling a neoplasia, tumour or cancer in a checkpoint inhibitor refractory subject, wherein said method comprises simultaneously, separately or sequentially administering to the subject (i) one or more checkpoint inhibitors, selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3 and combinations thereof, (ii) a non-viable, whole cell Mycobacterium, and (iii), administering one or more additional anticancer treatments or agents, selected from: adoptive cell therapy, surgical therapy, chemotherapy, radiation therapy, hormonal therapy, small molecule therapy such as metformin, receptor kinase inhibitor therapy, hyperthermia treatment, phototherapy, radioablation therapy, anti-angiogenic therapy, cytokine therapy, cryotherapy, biological therapy, HDAC inhibitor e.g. OKI-179, BRAF inhibitor, MEK inhibitor, EGFR inhibitor, VEGF inhibitor, PI3K delta inhibitor, PARP inhibitor, mTOR inhibitor, hypomethylating agents, oncolytic virus, TLR agonist including TLR2, 4, 7, 8 or 9 agonists, or TLR 5 agonists such as MRx0518 (4D Pharma), STING agonists (including MIW815 and SYNB1891), and cancer vaccines such as GVAX or CIMAvax, wherein said method results in enhanced therapeutic efficacy relative to administration of the one or more checkpoint inhibitors, co-stimulatory checkpoint therapy, one or more additional anticancer treatments or agents, or non-viable, whole cell Mycobacterium alone.

### DESCRIPTION OF THE DRAWINGS

The invention is described with reference to the following drawings, in which:

FIG. 1 shows the effect of a preparation of heat-killed Mycobacterium obuense (NCTC 13365) (IMM-101) with or without co-administration of a checkpoint inhibitor (ant-PD-L1 mAb), in a xenograft model of pancreatic cancer (KPC cells injected subcutaneously).

FIG. 2 shows the effect of a preparation of heat-killed Mycobacterium obuense (NCTC 13365) (IMM-101) with or without co-administration of a checkpoint inhibitor (anti-PD-1 mAb), in a syngeneic mouse model of breast cancer (EMT-6 cells injected subcutaneously) where the graph presents mean tumour volume against time+/−SE, as detailed in Example 3.

FIG. 3 shows the ratio of CD3+CD8+ cells and FoxP3 Treg cells infiltrating the B16F10 tumours as detailed in Example 2 in control mice, mice that have received anti-CTLA-4 treatment only or mice that have received combination treatment consisting of IMM-101 and anti-CTLA-4. We found a significant increase (Anova p<0.05) in this group. Lower graph shows the ratio of CD3+CD8+ cells and FoxP3 Treg cells infiltrating the tumours in control mice, mice that have received anti-PD-1 treatment only or mice that have received combination treatment consisting of IMM-101 and anti-PD-1.

FIG. 4 shows a schematic of study investigating the effect of anti-PD-1 antibody or anti-PD-1 antibody with IMM-101, in a mouse model of breast cancer using EMT-6 cell line, as detailed in Example 3.

FIG. 5 shows the impact on the change in tumour volume of vehicle, anti-PD-1 antibody or a combination of anti-PD-1 antibody with IMM-101, in a mouse model of breast cancer using EMT-6 cell line as detailed in Example 3.

FIG. 6 shows the impact on CD8/Treg ratio following administration of vehicle, anti-PD-1 antibody or anti-PD-1 antibody with IMM-101, in a mouse model of breast cancer using EMT-6 cell line as detailed in Example 3.

FIG. 7 shows the impact on INF-gamma/IL-10 ratio following administration of vehicle, anti-PD-1 antibody or anti-PD-1 antibody with IMM-101, in a mouse model of breast cancer using EMT-6 cell line, as detailed in Example 3.

FIG. 8 presents a schematic of study employing wild type (WT) and Batf−/− mice and graphical data on the influence of CD103+ DCs on INF-gamma release in WT and Batf−/− mice following s.c. injection of IMM-101.

FIG. 9 shows the effect of a preparation of heat-killed Mycobacterium obuense (NCTC 13365) (IMM-101), administered subcutaneously adjacent to the tumour, with or without co-administration of a checkpoint inhibitor (anti-PD-1 mAb), in a mouse model of a checkpoint resistant melanoma (B16F10) where the graph shows the effect on mean tumour volume+/−SE over time.

FIG. 10 is as FIG. 9 but where the graph shows the effect on mean tumour volume without SE (no error bars).

FIG. 11 is as FIG. 9 but where the graph shows the effect on mean tumour volume+/−SE up to study day 16.

FIG. 12 is as FIG. 9 but where the graph shows the effect on median tumour volume.

FIG. 13 is as FIG. 9 but where the graph shows the effect on median tumour volume up to study day 16.

FIG. 14 is as FIG. 9 but where the graph is a Kaplan-Meier survival graph.

FIG. 15 shows the effect of a preparation of heat-killed Mycobacterium obuense (NCTC 13365) (IMM-101), administered subcutaneously adjacent to the tumour, with or without co-administration of a checkpoint inhibitor (anti-PD-1 mAb), in a mouse model of a checkpoint resistant pancreatic cancer (Pan02) where the graph shows the effect on mean tumour volume+/−SE over time.

FIG. 16 is as FIG. 15 but where the graph shows the effect on mean tumour volume without SE (no error bars).

FIG. 17 is as FIG. 15 but where the graph shows the effect on mean tumour volume+/−SE up to study day 37.

FIG. 18 is as FIG. 15 but where the graph shows the effect on median tumour volume.

FIG. 19 is as FIG. 15 but where the graph shows the effect on median tumour volume up to study day 37.

### DETAILED DESCRIPTION OF THE INVENTION

The invention provides a method for treating, reducing, inhibiting or controlling a neoplasia, tumour or cancer in a checkpoint refractory subject involving administering a non-viable, whole cell Mycobacterium and one or more checkpoint inhibitors. It is based upon the surprising discovery that administration of a whole cell heat-killed Mycobacterium in combination with an anti-PD-L1 antibody (a checkpoint inhibitor), in a checkpoint inhibitor resistant animal model, results in synergistic anti-tumour activity and/or antitumor activity that is more potent than administration of the Mycobacterium or anti-PD-L1 antibody alone. Further, treatment with Mycobacterium has been shown to improve cytotoxic T-lymphocyte activity in a number of animal models with different cancer cell lines. This improved activity is believed to help reduce the innate and/or primary or adaptive resistance to checkpoint inhibitor therapy.

Accordingly, whilst some combination therapies are known in the art to reverse or reduce innate and/or primary or adaptive resistance, the invention disclosed herein provides combination therapies which are optimised to improve therapeutic efficacy and thus responses in a greater proportion of checkpoint refractory patients.

In order that the present invention may be more readily understood, certain terms are first defined. Additional definitions are set forth throughout the detailed description.

A “checkpoint inhibitor” is an agent which acts on surface proteins which are members of either the TNF receptor or B7 superfamilies, including agents which bind to negative co-stimulatory (co-inhibitory) molecules selected from CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3, and/or their respective ligands, including PD-L1. A “blocking agent” is an agent which either binds to the above negative co-stimulatory molecules and/or their respective ligands. “Checkpoint inhibitor” and “blocking agent” are used interchangeably throughout.

A non-viable, whole cell Mycobacterium as defined according to the present invention, is a component which stimulates innate and type-1 immunity, including Th1 and macrophage activation/polarization and cytotoxic cell activity, as well as independently down-regulating inappropriate anti-Th2 responses via immunoregulatory mechanisms.

The terms “tumour,” “cancer” and “neoplasia” are used interchangeably and refer to a cell or population of cells whose growth, proliferation or survival is greater than growth, proliferation or survival of a normal counterpart cell, e.g. a cell proliferative or differentiative disorder. Typically, the growth is uncontrolled. The term “malignancy” refers to invasion of nearby tissue. The term “metastasis” refers to spread or dissemination of a tumour, cancer or neoplasia to other sites, locations or regions within the subject, in which the sites, locations or regions are distinct from the primary tumour or cancer.

The terms “Programmed Death 1,” “Programmed Cell Death 1,” “Protein PD-1,” “PD-1,” and “PD1,” are used interchangeably, and include variants, isoforms, species homologs of human PD-1, and analogs having at least one common epitope with PD-1.

The terms “OX40”, “CD137” and “OX-40” are used interchangeably, and include variants, isoforms, species homologs of human OX40, and analogs having at least one common epitope with OX40.

The terms “GITR” and “Glucocorticoid-Induced TNFR family Related Gene” are used interchangeably, and include variants, isoforms, species homologs of human GITR, and analogs having at least one common epitope with GITR.

The terms “CD137” and “4-1BB” are used interchangeably, and include variants, isoforms, species homologs of human CD137, and analogs having at least one common epitope with CD137.

The terms “B7-H3” and “CD276” are used interchangeably, and include variants, isoforms, species homologs of human B7-H3, and analogs having at least one common epitope with B7-H3.

The terms “B7-H4” and “VTCN1” are used interchangeably, and include variants, isoforms, species homologs of human B7-H4, and analogs having at least one common epitope with B7-H4.

The terms “A2AR” and “Adenosine A2A receptor” are used interchangeably, and include variants, isoforms, species homologs of human A2AR, and analogs having at least one common epitope with A2AR.

The terms “IDO” and “Indoleamine 2,3-dioxygenase” are used interchangeably, and include variants, isoforms, species homologs of human IDO, and analogs having at least one common epitope with IDO.

The terms “cytotoxic T lymphocyte-associated antigen-4,” “CTLA-4,” “CTLA4,” and “CTLA-4 antigen” are used interchangeably, and include variants, isoforms, species homologs of human CTLA-4, and analogs having at least one common epitope with CTLA-4.

As used herein, “sub-therapeutic dose” means a dose of a therapeutic compound (e.g., an antibody) or duration of therapy which is lower than the usual or typical dose of the therapeutic compound or therapy of shorter duration, when administered alone for the treatment of cancer. Typical doses of known therapeutic compounds are known to those skilled in the art or can be determined through routine experimental work.

The term “therapeutically effective amount” is defined as an amount of a checkpoint inhibitor, in combination with a non-viable, whole cell Mycobacterium, that preferably results in a decrease in severity of cancer disease symptoms, an increase in frequency and duration of cancer disease symptom-free periods, or a prevention of impairment or disability due to the disease affliction. The terms “effective amount” or “pharmaceutically effective amount” refer to a sufficient amount of an agent to provide the desired biological or therapeutic result. That result can be reduction, amelioration, palliation, lessening, delaying, and/or alleviation of one or more of the signs, symptoms, or causes of a cancer or any other desired alteration of a biological system. In reference to cancer, an effective amount may comprise an amount sufficient to cause a tumour to shrink and/or to decrease the growth rate of the tumour (such as to suppress tumour growth) or to prevent or delay other unwanted cell proliferation. In some embodiments, an effective amount is an amount sufficient to delay development, or prolong survival or induce stabilisation of the cancer or tumour. Preferably, therapeutic efficacy is measured by a decrease or stabilisation of tumour size of one or more said tumours, as defined by RECIST 1.1, including stable diseases (SD), a complete response (CR) or partial response (PR) of the target tumour; and/or stable disease (SD) or complete response (CR) of one or more non-target tumours.

In some embodiments, a therapeutically effective amount is an amount sufficient to prevent or delay recurrence. A therapeutically effective amount can be administered in one or more administrations. The therapeutically effective amount of the drug or combination may result in one or more of the following: (i) reduce the number of cancer cells; (ii) reduce tumour size; (iii) inhibit, retard, slow to some extent and preferably stop cancer cell infiltration into peripheral organs; (iv) inhibit (i.e., slow to some extent and preferably stop) tumour metastasis; (v) inhibit tumour growth; (vi) prevent or delay occurrence and/or recurrence of tumour; and/or (vii) relieve to some extent one or more of the symptoms associated with the cancer. For example, for the treatment of tumours, a “therapeutically effective dosage” may induce tumour shrinkage by at least about 5% relative to baseline measurement, such as at least about 10%, or about 20%, or about 60% or more. The baseline measurement may be derived from untreated subjects.

A therapeutically effective amount of a therapeutic compound can decrease tumour size, or otherwise ameliorate symptoms in a subject. One of ordinary skill in the art would be able to determine such amounts based on such factors as the subject's size, the severity of the subject's symptoms, and the particular composition or route of administration selected.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells.

The term “antibody” as referred to herein includes whole antibodies and any antigen-binding fragment (i.e., “antigen-binding portion”) or single chains thereof.

The term “antigen-binding portion” of an antibody (or simply “antibody portion”), as used herein, refers to one or more fragments of an antibody that retain the ability to specifically bind to a receptor and its ligand (e.g., PD-1). including: (i) a Fab fragment, (ii) a F(ab′) 2 fragment; (iii) a Fd fragment consisting of the VH and CHI domains; (iv) a Fv fragment, (v) a dAb fragment which consists of a VH domain; and (vi) an isolated complementarity determining region (CDR). Single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences.

The term “humanized antibody” is intended to refer to antibodies in which CDR sequences derived from the germline of another mammalian species, such as a mouse, have been grafted onto human framework sequences. Additional framework region modifications may be made within the human framework sequences.

In addition to antibodies, other biological molecules may act as checkpoint inhibitors, including peptides having binding affinity to the appropriate target.

The term “treatment” or “therapy” refers to administering an active agent with the purpose to cure, heal, alleviate, relieve, alter, remedy, ameliorate, improve, or affect a condition (e.g., a disease), the symptoms of the condition, or to prevent or delay the onset of the symptoms, complications, biochemical indicia of a disease, or otherwise arrest or inhibit further development of the disease, condition, or disorder in a statistically significant manner.

As used herein, the term “subject” is intended to include human and non-human animals. Preferred subjects include human patients in need of enhancement of an immune response. The methods are particularly suitable for treating human patients having a disorder that can be treated by augmenting the T-cell mediated immune response. In a particular embodiment, the methods are particularly suitable for treatment of cancer cells in vivo.

As used herein, the term “checkpoint inhibitor refractory patient” refers to a patient identified as non-responsive to checkpoint inhibitor therapy. A refractory patient may exhibit an innate (primary) resistance to checkpoint inhibitor therapy. Innate resistance may be demonstrated by a lack of response or an insufficient response to said checkpoint inhibitor therapy for at least about 8 weeks, or 12 weeks from the first dose. A refractory patient may exhibit an acquired (secondary) resistance to checkpoint inhibitor therapy. Acquire resistance may be demonstrated by an initial response to said checkpoint therapy but with a subsequent relapse and progression of one or more tumours. Checkpoint inhibitor refractory patients can be non-responsive to any checkpoint inhibitor, non-limiting example include CTLA-4, PD-1, PD-L1, PD-L2, B7-H3, B7-H4, B7-H6, A2AR, IDO, TIM-3, BTLA, VISTA, LAG-3 inhibitors, or combinations thereof.

As used herein, the terms “concurrent administration” or “concurrently” or “simultaneous” mean that administration occurs on the same day. The terms “sequential administration” or “sequentially” or “separate” mean that administration occurs on different days.

“Simultaneous” administration, as defined herein, includes the administration of the non-viable, whole cell Mycobacterium and agent or procedure comprising checkpoint inhibitor therapy and/or co-stimulatory checkpoint therapy, and/or one or more additional anticancer treatments or agents, within about 2 hours or about 1 hour or less of each other. Preferably “simultaneous” administration refers to wherein the non-viable, whole cell Mycobacterium and agent or procedure comprising checkpoint inhibitor therapy and/or co-stimulatory checkpoint therapy, and/or one or more additional anticancer treatments or agents are administered at the same time.

“Separate” administration, as defined herein, includes the administration of the non-viable, whole cell Mycobacterium and agent or procedure comprising checkpoint inhibitor therapy, and/or co-stimulatory checkpoint therapy, and/or one or more additional anticancer treatments or agents, more than about 12 hours, or about 8 hours, or about 6 hours or about 4 hours or about 2 hours apart.

“Sequential” administration, as defined herein, includes the administration of the non-viable, whole cell Mycobacterium and agent or procedure comprising checkpoint inhibitor therapy and/or co-stimulatory checkpoint therapy, and/or one or more additional anticancer treatments or agents, each in multiple aliquots and/or doses and/or on separate occasions. The non-viable, whole cell Mycobacterium may be administered to the patient after before and/or after administration of the checkpoint inhibitor, and/or co-stimulatory checkpoint therapy, and/or one or more additional anticancer treatments or agents. Alternatively, the non-viable, whole cell Mycobacterium is continued to be applied to the patient after treatment with a checkpoint inhibitor and/or co-stimulatory checkpoint therapy, and/or one or more additional anticancer treatments or agents.

The use of the alternative (e.g., “or”) should be understood to mean either one, both, or any combination thereof of the alternatives. As used herein, the indefinite articles “a” or “an” should be understood to refer to “one or more” of any recited or enumerated component.

As used herein, “about” means within an acceptable error range for the particular value as determined by one of ordinary skill in the art, which will depend in part on how the value is measured or determined, i.e., the limitations of the measurement system. For example, “about” can mean within 1 or more than 1 standard deviation per the practice in the art. Alternatively, “about” can mean a range of up to 20%. When particular values are provided in the application and claims, unless otherwise stated, the meaning of “about” should be assumed to be within an acceptable error range for that particular value.

M. vaccae and M. obuense have been shown to induce a complex immune response in the host. Treatment with these preparations will stimulate innate and type-1 immunity, including Th1 and macrophage activation and cytotoxic cell activity. They also independently down-regulate inappropriate Th2 responses via immunoregulatory mechanisms. It has been shown in experiments with mouse and human immune cells that IMM-101 (a non-viable, whole cell M. obuense) is a strong activator of antigen presenting macrophages and dendritic cells (DCs) and that the DC activation leads to a typical Type 1 immune response, with formation and activation of CD4+T-helper 1 lymphocytes (Th1s) and CD8+ cytotoxic T lymphocytes (CTLs) and increased production of the cytokine interferon-γ (IFN-γ) in the lymph nodes in which IMM-101 activated DCs are present.

In addition, other experiments have shown that IMM-101 also increases the number and activation of natural killer cells (NKs) and T cells expressing gamma/delta receptors (γδ-T cells). Th1s and CTLs require tumour cells to express specific tumour-associated antigens (TAAs) for their attack, whereas NK and γδ-T cells do not require the presence of such TAAs to kill tumour cells. These four different immune cells work in concert to form an effective anti-tumour response. In relation to cancer, it is likely that the formation of IFN-γ producing CTLs is the most important result from IMM-101 treatment, since the observed anti-tumour effect of IMM-101 could be completely abrogated by the depletion of CD8+ T cells in a pancreas cancer model.

IMM-101's ability to activate macrophages may not only assist in the activation of DCs through the release of pro-inflammatory macrophage-derived cytokines (such as IL-12 required for skewing DCs into Type 1 immune responses), but may also be of importance for changing tumour associated immunosuppressive type 2 macrophages into tumour aggressive type 1 macrophages. This latter feature was shown for a similar heat-killed mycobacterium, M. indicus pranii.

An important feature of IMM-101 is its ability to activate and mature DCs into a sub-class of dendritic cells known as cDC1s (i.e. DCs that are required for Type 1 immune responses). It has been shown that activation of sufficient numbers of cDC1s is a prerequisite for CPIs to be effective.

It is generally believed that a Type 1 immune response resulting in INF-γ producing Th1s and CTLs, which specifically attack TAAs-expressing tumour cells, and activated NK and γδ-T cells, which attack tumours through other mechanisms, is the body's main mechanism and a pre-requisite for an effective anti-cancer response and should therefore be at the core of any immune-mediated cancer treatment—preclinical data show that IMM-101 is capable of stimulating such required Type 1 immune responses.

The impact of IMM-101 on DC priming has been studied in vitro and it was found that IMM-101 displayed a dose-dependent ability to induce phenotypic activation and cytokine production for both human and murine DCs. For example, GM-CSF derived murine DC displayed a dose dependent response to IMM-101, with elevated membrane expression of CD80, CD86, CD40 and MHC II and increased production of IL-6, IL-12p40 and nitric oxide, which are all molecules that are essential for effective antigen-dependent activation of T cells. Moreover, human monocyte-derived DCs showed a similar response to IMM-101, with up-regulation of CD80, CD86 and MHC II and secretion of a number of relevant cytokines, showing clear activation of DCs. Exposure to IMM-101 in vitro also showed that IMM-101 functionally affects the DCs by enhancing their ability to process and present antigen.

In vivo experiments have shown that IMM-101 activated DCs are able to activate CD8+ and CD4+ T cells and promote secretion of IFN-γ following re-stimulation of draining lymph node cell preparations, 7 days after subcutaneous adoptive transfer of IMM-101 (in vitro) activated GM-CSF derived murine DCs into naïve recipient mice.

Disclosed herein, the combination of several different checkpoint inhibitors (CPIs) with a non-viable whole cell Mycobacterium (IMM-101) has been investigated in animal models using three different cancer cell lines (breast, pancreatic and a checkpoint resistant form of melanoma, B16F10. In these experiments, CPI (or IMM-101) treatment alone was only moderately to poorly effective in controlling growth of the primary tumour, but the combination of a CPI with IMM-101 showed a positive trend in breast cancer and was highly effective in reducing tumour volume in pancreas cancer. An increased CD8+/Treg ratio was found within the breast tumour and melanoma. An increased IFN-γ/IL-10 (IL-10 is a cytokine produced by Tregs) ratio was also found in the spleen of the breast cancer model. These increased ratios are highly suggestive of an improvement in the CTL activity and a reduction in the immune suppression activity, which is expected to result in reducing or inhibiting primary and/or secondary resistance to checkpoint inhibitor therapy in a human or animal subject.

In one aspect of the present invention the non-viable, whole cell Mycobacterium comprises a whole cell, non-pathogenic heat-killed Mycobacterium. Examples of mycobacterial species for use in the present invention include M. vaccae, M. thermoresistibile, M. flavescens, M. duvalii, M. phlei, M. obuense, M. parafortuitum, M. sphagni, M. aichiense, M. rhodesiae, M. neoaurum, M. chubuense, M. tokaiense, M. komossense, M. aurum, M. w, M. tuberculosis, M. microti; M. africanum; M. kansasii, M. marinum; M. simiae; M. gastri; M. nonchromogenicum; M. terrae; M. triviale; M. gordonae; M. scrofulaceum; M. paraffinicum; M. intracellulare; M. avium; M. xenopi; M. ulcerans; M. diemhoferi, M. smegmatis; M. thamnopheos; M. flavescens; M. fortuitum; M. peregrinum; M. chelonei; M. paratuberculosis; M. leprae; M. lepraemurium and combinations thereof.

The non-viable, whole cell Mycobacterium is preferably selected from M. vaccae, M. obuense, M. parafortuitum, M. aurum, M. indicus pranii, M. phlei and combinations thereof. More preferably the non-viable, whole cell Mycobacterium is a rough variant.

The amount of Mycobacterium administered to the patient is sufficient to elicit an immune response in the patient such that the patient's immune system is able to mount an effective immune response to the cancer or tumour. In certain embodiments of the invention, there is provided a containment means comprising the effective amount of Mycobacterium for use in the present invention, which typically may be from 10³ to 10¹¹ organisms, preferably from 10⁴ to 10¹⁰ organisms, more preferably from 10⁶ to 10¹⁰ organisms, and even more preferably from 10⁶ to 10⁹ organisms. Most preferably the amount of Mycobacterium for use in the present invention is from 10⁷ to 10⁹ cells or organisms. Typically, the composition according to the present invention may be administered at a dose of from 10⁸ to 10⁹ cells for human and animal use. Alternatively the dose is from 0.01 mg to 5 mg or 0.01 mg to 5 mg organisms, preferably 0.1 mg to 2 mg or 0.1 mg to 2 mg organisms, more preferably the dose is approximately 1 mg or 1 mg organisms. The dose may be prepared as either a suspension or dry preparation.

M. indicus pranii, M. vaccae and M. obuense are particularly preferred.

The present invention may be used to treat a neoplastic disease, such as solid or non-solid cancers. As used herein, “treatment” encompasses the prevention, reduction, control and/or inhibition of a neoplastic disease. Such diseases include a sarcoma, carcinoma, adenocarcinoma, melanoma, myeloma, blastoma, glioma, lymphoma or leukemia. Exemplary cancers include, for example, carcinoma, sarcoma, adenocarcinoma, melanoma, neural (blastoma, glioma), mesothelioma and reticuloendothelial, lymphatic or haematopoietic neoplastic disorders (e.g., myeloma, lymphoma or leukemia). In particular aspects, a neoplasm, tumour or cancer includes a lung adenocarcinoma, lung carcinoma, diffuse or interstitial gastric carcinoma, colon adenocarcinoma, prostate adenocarcinoma, esophagus carcinoma, breast carcinoma, pancreas adenocarcinoma, ovarian adenocarcinoma, adenocarcinoma of the adrenal gland, adenocarcinoma of the endometrium or uterine adenocarcinoma.

Neoplasia, tumours and cancers include benign, malignant, metastatic and non-metastatic types, and include any stage (I, II, III, IV or V) or grade (G1, G2, G3, etc.) of neoplasia, tumour, or cancer, or a neoplasia, tumour, cancer or metastasis that is progressing, worsening, stabilized or in remission. Cancers that may be treated according to the invention include but are not limited to: bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus. In addition, the cancer may specifically be of the following histological type, though it is not limited to the following: neoplasm, malignant carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumour, malignant bronchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma with squamous metaplasia; thymoma, malignant ovarian stromal tumour, malignant thecoma, malignant granulosa cell tumour, malignant androblastoma, malignant Sertoli cell carcinoma; Leydig cell tumour, malignant lipid cell tumour, malignant paraganglioma, malignant extra-mammary paraganglioma, malignant pheochromocytoma; glomangiosarcoma; malignant melanoma; amelanotic melanoma; superficial spreading melanoma; malignant melanoma in giant pigmented nevus; metastatic melanoma, Lentigo Maligna, Lentigo Maligna Melanoma, Nodular Melanoma, Acral Lentiginous Melanoma, desmoplastic Melanoma, epithelioid cell melanoma; blue nevus, malignant sarcoma; fibrosarcoma; fibrous histiocytoma, malignant myxosarcoma; liposarcoma; leiomyosarcoma; rhabdomyosarcoma; embryonal rhabdomyosarcoma; alveolar rhabdomyosarcoma; stromal sarcoma; mixed tumour; Mullerian mixed tumour; nephroblastoma; hepatoblastoma; carcinosarcoma; mesenchymoma, malignant Brenner tumour, malignant phyllodes tumour, malignant synovial sarcoma; mesothelioma, malignant dysgerminoma; embryonal carcinoma; teratoma, malignant; struma ovarii, malignant; choriocarcinoma; mesonephroma, malignant hemangiosarcoma; hemangioendothelioma, malignant Kaposi's sarcoma; hemangiopericytoma, malignant lymphangiosarcoma; osteosarcoma; juxtacortical osteosarcoma; chondrosarcoma; chondroblastoma, malignant mesenchymal chondrosarcoma; giant cell tumour of bone; Ewing's sarcoma; odontogenic tumour, malignant ameloblastic odontosarcoma; ameloblastoma, malignant ameloblastic fibrosarcoma; pinealoma, malignant chordoma; glioma, malignant ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumour; meningioma, malignant neurofibrosarcoma; neurilemmoma, malignant granular cell tumour, malignant lymphoma; Hodgkin's disease; Hodgkin's paragranuloma; malignant lymphoma, small lymphocytic malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Preferably, the cancer is selected from prostate cancer, liver cancer, renal cancer, lung cancer, breast cancer, colorectal cancer, pancreatic cancer, brain cancer, hepatocellular cancer, lymphoma, leukaemia, gastric cancer, cervical cancer, ovarian cancer, thyroid cancer, melanoma, head and neck cancer, skin cancer and soft tissue sarcoma and/or other forms of carcinoma. The tumour may be metastatic or a malignant tumour.

More preferably, the cancer is pancreatic, colorectal, prostate, skin, ovarian or lung cancer.

In an embodiment of the invention, there is provided a non-viable, whole-cell Mycobacterium for use in the treatment, reduction, inhibition or control of one or more tumours in a checkpoint inhibitor refractory patient, wherein said checkpoint inhibitor refractory patient is intended to undergo checkpoint inhibition therapy simultaneously, separately or sequentially with administration of the Mycobacterium.

In an embodiment of the invention, there is provided a non-viable, whole-cell Mycobacterium for use in the treatment, reduction, inhibition or control of one or more tumours in a checkpoint inhibitor refractory patient, wherein said checkpoint inhibitor refractory patient is intended to undergo checkpoint inhibition therapy simultaneously, separately or sequentially with administration of the Mycobacterium, wherein said checkpoint inhibition therapy comprises administration of one or more blocking agents, selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3 and combinations thereof.

In an embodiment of the invention, the checkpoint inhibition therapy comprises administration of a sub-therapeutic amount and/or duration of said one or more blocking agents.

In an embodiment of the invention, the one or more blocking agents are selected from ipilimumab, nivolumab, pembrolizumab, azetolizumab, durvalumab, tremelimumab, spartalizumab, avelumab, sintilimab, toripalimab, MGA012, MGD013, MGD019, enoblituzumab, MGD009, MGC018, MEDI0680, PDR001, FAZ053, TSR022, MBG453, relatlinab (BMS986016), LAG525, IMP321, REGN2810 (cemiplimab), REGN3767, pexidartinib, LY3022855, FPA008, BLZ945, GDC0919, epacadostat, indoximid, BMS986205, CPI-444, MEDI9447, PBF509, lirilumab and combinations thereof.

In an embodiment of the invention, the one or more blocking agents are preferably ipilimumab and/or nivolumab.

In an embodiment of the invention, the checkpoint inhibitor therapy comprises administration of one or more blocking agents, selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3 and combinations thereof, in combination with a non-viable, whole cell, Mycobacterium, to reduce or inhibit metastasis of a primary tumour or cancer to other sites, or the formation or establishment of metastatic tumours or cancers at other sites distal from the primary tumour or cancer thereby inhibiting or reducing tumour or cancer relapse or tumour or cancer progression, preferably in a checkpoint inhibitor refractory patient.

In an embodiment of the invention, there is provided a non-viable, whole-cell Mycobacterium for use in the treatment, reduction, inhibition or control of one or more tumours in a checkpoint inhibitor refractory patient, wherein said checkpoint inhibitor refractory patient is intended to undergo checkpoint inhibition therapy simultaneously, separately or sequentially with administration of the Mycobacterium, wherein said checkpoint inhibition therapy comprises administration of one or more blocking agents, selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3 and combinations thereof, with the potential to elicit potent and durable immune responses with enhanced therapeutic benefit compared to either therapy alone, preferably as measured by a decrease or stabilisation of tumour size of one or more said tumours, as defined by RECIST 1.1, including stable diseases (SD), a complete response (CR) or partial response (PR) of the target tumour; and/or stable disease (SD) or complete response (CR) of one or more non-target tumours.

In an embodiment of the invention, there is provided a non-viable, whole-cell Mycobacterium for use in the manufacture of a medicament for the treatment of cancer in a checkpoint inhibitor refractory patient, wherein said checkpoint inhibitor refractory patient is intended to undergo checkpoint inhibition therapy simultaneously, separately or sequentially with administration of the Mycobacterium.

In a further embodiment of the invention, there is provided a combination therapy for treating cancer in a checkpoint inhibitor refractory patient, comprising a non-viable, whole-cell Mycobacterium which; (i) stimulates innate and type-1 immunity, including Th1 and macrophage activation and cytotoxic cell activity, and, (ii) independently down-regulates inappropriate Th2 responses via immunoregulatory mechanisms; and, a checkpoint inhibitor, optionally wherein the Mycobacterium is selected from M. vaccae, M. obuense or M. indicus pranii.

In a further embodiment of the invention, there is provided a combination therapy for treating cancer in a checkpoint inhibitor refractory patient, comprising a non-viable, whole-cell Mycobacterium which mediates any combination of at least one of the following immunostimulatory effects on immunity: (i) increasing immune response, (ii) increasing T cell activation, (iii) increasing cytotoxic T cell activity, (iv) increasing NK cell activity, (v) increasing Th17 activity, (vi) alleviating T-cell suppression, (vii) increasing pro-inflammatory cytokine secretion, (viii) increasing IL-2 secretion; (ix) increasing interferon-γ production by T-cells, (x) increasing Th1 response, (xi) decreasing Th2 response, (xii) decreasing or eliminating at least one of regulatory T cells (Tregs), myeloid derived suppressor cells (MDSCs), iMCs, mesenchymal stromal cells, TIE2-expressing monocytes, (xiii) reducing regulatory cell activity and/or the activity of one or more of myeloid derived suppressor cells (MDSCs), iMCs, mesenchymal stromal cells, TIE2-expressing monocytes, (xiv) decreasing or eliminating M2 macrophages, (xv) reducing M2 macrophage pro-tumorigenic activity, (xvi) decreases or eliminates N2 neutrophils, (xvii) reduces N2 neutrophils pro-tumorigenic activity, (xviii) reducing inhibition of T cell activation, (xix) reducing inhibition of CTL activation, (xx) reducing inhibition of NK cell activation, (xxi) reversing T cell exhaustion, (xxii) increasing T cell response, (xxiii) increasing activity of cytotoxic cells, (xxiv) stimulating antigen-specific memory responses, (xxv) eliciting apoptosis or lysis of cancer cells, (xxvi) stimulating cytotoxic or cytostatic effect on cancer cells, (xxvii) inducing direct killing of cancer cells, and/or (xxviii) inducing complement dependent cytotoxicity and/or (xxix) inducing antibody dependent cell-mediated cytotoxicity.

In a further embodiment of the invention, there is provided a combination therapy for treating cancer in a checkpoint inhibitor refractory patient, comprising a non-viable, whole-cell Mycobacterium which promotes CTL activity, wherein CTL activity includes the secretion of one or more proinflammatory cytokines and/or CTL mediated killing of target cells; and/or which promotes CD4+ T cell activation and/or CD4+ T cell proliferation and/or CD4+ T cell mediated cell depletion; and/or which promotes CD8+ T cell activation and/or CD8+ T cell proliferation and/or CD8+ T cell mediated cell depletion; and/or which enhances NK cell activity, and/or NK cell proliferation and/or NK cell mediated cell depletion, wherein enhanced NK cell activity includes increased depletion of target cells and/or proinflammatory cytokine release; and/or upregulation or stimulation of CD103+CD141+ DCs; and/or which decreases or eliminates the differentiation, proliferation and/or activity of regulatory cells (Tregs), and/or the differentiation, proliferation, infiltration and/or activity of myeloid derived suppressor cells (MDSCs); and/or which decreases or eliminates the infiltration of inducible Tregs (iTregs) into a target site.

In an embodiment of the invention, there is provided a non-viable, whole-cell Mycobacterium for use in the treatment, reduction, inhibition or control of one or more tumours in a checkpoint inhibitor refractory patient, wherein said checkpoint inhibitor refractory patient is intended to undergo checkpoint inhibition therapy simultaneously, separately or sequentially with administration of the Mycobacterium, further comprising co-stimulatory checkpoint therapy, simultaneously, separately or sequentially with administration of the Mycobacterium, wherein said co-stimulatory checkpoint therapy comprises administration of one or more binding agents selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CD27, CD28, CD40, CD122, CD137, OX40, GITR, ICOS and combinations thereof.

In an embodiment of the invention, the co-stimulatory checkpoint therapy comprises administration of one or more binding agents selected from utomilumab, urelumab, MOXR0916. PF04518600, MEDI0562, GSK3174988, MEDI6469. RO7009789, CP870893, BMS986156, GWN323, JTX-2011, varlilumab, MK-4166, NKT-214 and combinations thereof.

In an embodiment of the invention, administration of said non-viable whole-cell Mycobacterium is prior to and/or after the checkpoint inhibition therapy and/or the co-stimulatory checkpoint therapy.

In a further embodiment of the invention, there is provided a non-viable, whole-cell Mycobacterium for use in the treatment, reduction, inhibition or control of one or more tumours in a checkpoint inhibitor refractory patient, wherein said checkpoint inhibitor refractory patient is intended to undergo checkpoint inhibition therapy simultaneously, separately or sequentially with administration of the Mycobacterium, further comprising administering one or more additional anticancer treatments or agents, simultaneously, separately or sequentially with administration of the Mycobacterium, and/or checkpoint inhibition therapy and/or the co-stimulatory checkpoint therapy.

In a further embodiment of the invention, the one or more additional anticancer treatments or agents is selected from: adoptive cell therapy, surgical therapy, chemotherapy, radiation therapy, hormonal therapy, small molecule therapy such as metformin, receptor kinase inhibitor therapy, hyperthermia treatment, phototherapy, radioablation therapy, anti-angiogenic therapy, cytokine therapy, cryotherapy, biological therapy, HDAC inhibitor e.g. OKI-179, BRAF inhibitor, MEK inhibitor, EGFR inhibitor, VEGF inhibitor, P13K delta inhibitor, PARP inhibitor, mTOR inhibitor, hypomethylating agents, oncolytic virus, TLR agonist including TLR2, 3, 4, 7, 8 or 9 agonists, or TLR 5 agonists such as MRx0518 (4D Pharma), STING agonists (including MIW815 and SYNB1891), and cancer vaccines such as GVAX or CIMAvax.

In another embodiment of the invention, the one or more additional anticancer treatments results in immunogenic cell death therapy, as described in WO2013107998. This therapy results in the induction of tumour immunogenic cell death, including apoptosis (type 1), autophagy (type 2) and necrosis (type 3), whereupon there is a release of tumour antigens that are able to both induce immune responses, including activation of cytotoxic CD8+ T cells and NK cells and to act as targets, including rendering antigens accessible to Dendritic Cells. The immunogenic cell death therapy may be carried out at sub-optimal levels, i.e. non-curative therapy such that it is not intended to fully remove or eradicate the tumour, but nevertheless results in some tumour cells or tissue becoming necrotic. The skilled person will appreciate the extent of therapy required in order to achieve this, depending on the technique used, age of the patient, status of the disease and particularly size and location of tumour or metastases. Particularly preferred treatments include: microwave irradiation, targeted radiotherapy such as stereotactic ablative radiation (SABR), embolisation, cryotherapy, ultrasound, high intensity focused ultrasound, cyberknife, hyperthermia, radiofrequency ablation, cryoablation, electrotome heating, hot water injection, alcohol injection, embolization, radiation exposure, photodynamic therapy, laser beam irradiation, and combinations thereof.

In a further embodiment of the invention, the TLR agonists include MRx0518 (4D Pharma), mifamurtide (Mepact), Krestin (PSK), IMO-2125 (tilsotolimod), CMP-001, MGN-1703 (lefitolimod), entolimod, SD-101, GS-9620, imiquimod, resiquimod, MEDI4736, poly I:C, CPG7909, DSP-0509, VTX-2337 (motolimod), MEDI9197, NKTR-262, G100 or PF-3512676 and combinations thereof.

In a further embodiment of the invention, the chemotherapy comprises administration of one or more agents selected from: cyclophosphamide, methotrexate, 5-fluorouracil, doxorubicin, mustine, vincristine, procarbazine, prednisolone, bleomycin, vinblastine, dacarbazine, etoposide, cisplatin, epirubicin, capecitabine, leucovorin, folinic acid, carboplatin, oxaliplatin, gemcitabine, FOLFINROX, paclitaxel, pemetrexed, irinotecan and combinations thereof.

In a further embodiment of the invention, the one or more additional anticancer treatments or agents is administered intratumorally, intraarterially, intravenously, intravascularly, intrapleuraly, intraperitoneally, intratracheally, intranasally, pulmonarily, intrathecally, intramuscularly, endoscopically, intralesionally, percutaneously, subcutaneously, regionally, stereotactically, orally or by direct injection or perfusion.

In a preferred embodiment of the invention, the checkpoint inhibitor refractory patient exhibits an innate (primary) resistance to checkpoint inhibitor therapy or an acquired (secondary) resistance to checkpoint inhibitor therapy.

In a preferred embodiment of the invention, the checkpoint inhibitor refractory patient exhibits an innate (primary) resistance to checkpoint inhibitor therapy as demonstrated by a lack of response or an insufficient response to said checkpoint inhibitor therapy for at least about 8 weeks, or 12 weeks from the first dose.

In a preferred embodiment of the invention, the checkpoint inhibitor refractory patient exhibits an acquired (secondary) resistance to checkpoint inhibitor therapy as demonstrated by an initial response to said checkpoint therapy but with a subsequent relapse and progression of one or more tumours.

In yet another preferred embodiment of the invention, the checkpoint inhibitor refractory patient exhibits an innate (primary) resistance or an acquired (secondary) resistance to treatment with one or more CTLA-4, PD-1, PD-L1, PD-L2, B7-H3, B7-H4, B7-H6, A2AR, IDO, TIM-3, BTLA, VISTA, LAG-3 inhibitors.

In an embodiment of the invention, the checkpoint inhibition therapy and/or the co-stimulatory checkpoint therapy act synergistically with the Mycobacterium.

In an embodiment of the invention, there is provided a method of treating, reducing, inhibiting or controlling a neoplasia, tumour or cancer in a checkpoint inhibitor refractory subject, wherein said method comprises simultaneously, separately or sequentially administering to the subject, (i) one or more checkpoint inhibitors, selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3 and combinations thereof, and (ii) a non-viable, whole cell Mycobacterium, wherein said method results in enhanced therapeutic efficacy relative to administration of the one or more checkpoint inhibitors or non-viable, whole cell Mycobacterium alone, optionally wherein the checkpoint inhibition therapy comprises administration of a sub-therapeutic amount and/or duration of said one or more blocking agents.

In an embodiment of the invention, there is provided a method of treating, reducing, inhibiting or controlling a neoplasia, tumour or cancer in a checkpoint inhibitor refractory subject, wherein said method comprises simultaneously, separately or sequentially administering to the subject, (i) one or more checkpoint inhibitors selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CTLA-4, PD-1 or PD-L1, and combinations thereof, and (ii) a non-viable, whole cell Mycobacterium.

In an embodiment of the invention, there is provided a method of treating, reducing, inhibiting or controlling a neoplasia, tumour or cancer in a checkpoint inhibitor refractory subject, wherein said method comprises simultaneously, separately or sequentially administering to the subject, (i) one or more checkpoint inhibitors selected from ipilimumab, nivolumab, pembrolizumab, azetolizumab, durvalumab, tremelimumab, spartalizumab, avelumab, sintilimab, toripalimab, MGA012, MGD013, MGD019, enoblituzumab, MGD009, MGC018, MEDI0680, PDR001, FAZ053, TSR022, MBG453, relatlinab (BMS986016), LAG525, IMP321, REGN2810 (cemiplimab), REGN3767, pexidartinib, LY3022855, FPA008, BLZ945, GDC0919, epacadostat, indoximid, BMS986205, CPI-444, MEDI9447, PBF509. lirilumab and combinations thereof, and (ii) a non-viable, whole cell Mycobacterium.

In a preferred embodiment of the invention, the one or more checkpoint inhibitors are selected from ipilimumab and/or nivolumab.

In an embodiment of the invention, there is provided a method of treating, reducing, inhibiting or controlling a neoplasia, tumour or cancer in a checkpoint inhibitor refractory subject, wherein said one or more tumours is associated with a cancer selected from prostate cancer, liver cancer, renal cancer, lung cancer, breast cancer, colorectal cancer, breast cancer, pancreatic cancer, brain cancer, hepatocellular cancer, lymphoma, leukaemia, gastric cancer, cervical cancer, ovarian cancer, thyroid cancer, melanoma, carcinoma, head and neck cancer, skin cancer and soft tissue sarcoma, preferably wherein said one or more tumours is associated with pancreatic, colorectal, prostate, skin or ovarian cancer.

In a further embodiment of the invention, there is provided a method of treating, reducing, inhibiting or controlling a neoplasia, tumour or cancer in a checkpoint inhibitor refractory subject, wherein the method comprises simultaneously, separately or sequentially administering to the subject, (i) one or more checkpoint inhibitors (ii), a non-viable, whole cell Mycobacterium, and (iii), co-stimulatory checkpoint therapy, simultaneously, separately or sequentially with administration of the Mycobacterium, wherein said co-stimulatory checkpoint therapy comprises administration of one or more binding agents, selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CD27, CD28, CD40, CD122, CD137, OX40, GITR, ICOS and combinations thereof, wherein said method results in enhanced therapeutic efficacy relative to administration of the one or more checkpoint inhibitors, co-stimulatory checkpoint therapy, or non-viable, whole cell Mycobacterium alone, and optionally wherein said co-stimulatory checkpoint therapy comprises administration of a sub-therapeutic amount and/or duration of said binding agent.

In a further embodiment of the invention, the co-stimulatory checkpoint therapy comprises administration of one or more binding agents, selected from utomilumab, urelumab, MOXR0916. PF04518600, MEDI0562, GSK3174988, MEDI6469. RO7009789, CP870893, BMS986156, GWN323, JTX-2011, varlilumab, MK-4166, NKT-214 and combinations thereof.

In a further embodiment of the invention, there is provided a method of treating, reducing, inhibiting or controlling a neoplasia, tumour or cancer in a checkpoint inhibitor refractory subject, wherein the method comprises simultaneously, separately or sequentially administering to the subject, (i) one or more checkpoint inhibitors (ii), a non-viable, whole cell Mycobacterium, and (iii), co-stimulatory checkpoint therapy, simultaneously, separately or sequentially with administration of the Mycobacterium, wherein said co-stimulatory checkpoint therapy comprises administration of one or more binding agents, wherein said binding agent is an agonistic antibody, optionally wherein said method comprises administration of a sub-therapeutic amount and/or duration of said co-stimulatory checkpoint binding agent.

In yet a further embodiment of the invention, there is provided a non-viable, whole-cell Mycobacterium for use in the treatment, reduction, inhibition or control of one or more tumours in a checkpoint inhibitor refractory patient, wherein said checkpoint inhibitor refractory patient is intended to undergo checkpoint inhibition therapy simultaneously, separately or sequentially with administration of the Mycobacterium, wherein said checkpoint inhibition therapy comprises administration of two or more blocking agents, selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3 and combinations thereof, and optionally wherein said checkpoint inhibition therapy comprises administration of a sub-therapeutic amount and/or duration of said blocking agents.

In yet a further embodiment of the invention, there is provided a non-viable, whole-cell Mycobacterium for use in the treatment, reduction, inhibition or control of one or more tumours in a checkpoint inhibitor refractory patient, wherein said checkpoint inhibitor refractory patient is intended to undergo checkpoint inhibition therapy simultaneously, separately or sequentially with administration of the Mycobacterium, wherein said checkpoint inhibition therapy comprises administration of two or more blocking agents, wherein said two or more blocking agents are directed against any one of the following combinations: CTLA-4 and PD-1, CTLA-4 and PD-L1, PD-1 and LAG-3, or PD-1 and PD-L1.

Suitable specific combinations include: Durvalumab+tremelimumab, Nivolumab+ipilimumab, Pembrolizumab+ipilimumab, MEDI0680+durvalumab, PDR001+FAZ053, Nivolumab+TSR022, PDR001+MBG453, Nivolumab+BMS 986016, PDR001+LAG525, Pembrolizumab+IMP321, REGN2810 (cemiplimab)+REGN3767, and other suitable combinations.

In an embodiment of the invention, there is provided a non-viable, whole-cell Mycobacterium for use in the treatment, reduction, inhibition or control of one or more tumours in a checkpoint inhibitor refractory patient, wherein said checkpoint inhibitor refractory patient is intended to undergo checkpoint inhibition therapy simultaneously, separately or sequentially with administration of the Mycobacterium, further comprising co-stimulatory checkpoint therapy, simultaneously, separately or sequentially with administration of the Mycobacterium, directed against any one of the following combinations: CTLA-4 and CD40, CTLA-4 and OX40, CTLA-4 and IDO, OX-40 and PD-L1, PD-1 and OX-40, CD27 and PD-L1, PD-1 and CD137, PD-L1 and CD137, OX-40 and CD137, CTLA-4 and IDO, PD-1 and IDO, PD-L1 and IDO, PD! And A2AR, PD-L1 and A2AR, PD1 and GITR, PD-L1 and GITR, PD1 and ICOS, PD-L1 and ICOS, PD1 and CD27, PD-L1 and CD27, PD1 and CD122, PD-L1 and CD122, PD1 and CSF1R, PD-L1 and CSF1R, and other such suitable combinations.

Suitable specific combinations include: Avelumab+utomilumab, Nivolumab+urelumab, Pembrolizumab+utomilumab, Atezolimumab+MOXR0916±bevacizumab, Avelumab+PF-04518600, Durvalumab+MEDI0562, Pembrolizumab+GSK3174998, Tremelimumab+durvalumab+MEDI6469, Tremelimumab+MEDI0562, Utomilumab+PF-04518600, Atezolimumab+RO7009789, Tremelimumab+CP870893, Nivolumab+BMS986156, PDR001+GWN323, Nivolumab+JTX-2011, Atezolizumab+GDC0919, Ipilimumab+epacadostat, Ipilimumab+indoximid, Nivolumab+BMS986205, Pembrolizumab+epacadostat, Atezolizumab+CPI-444, Durvalumab+MEDI9447, PDR001+PBF509, Nivolumab+varlilumab, Atezolizumab+varlilumab, Nivolumab+NKTR-214, Durvalumab+Pexidartinib (PLX3397), Durvalumab+LY3022855, Nivolumab+FPA008, Pembrolizumab+Pexidartinib, PDR001+BLZ945, Tremelimumab+LY3022855.

In a further embodiment of the invention, there is provided a method of treating, reducing, inhibiting or controlling a neoplasia, tumour or cancer in a checkpoint inhibitor refractory subject, wherein the method comprises simultaneously, separately or sequentially administering to the subject, (i) one or more checkpoint inhibitors (ii), a non-viable, whole cell Mycobacterium, and (iii), co-stimulatory checkpoint therapy, simultaneously, separately or sequentially with administration of the Mycobacterium, further comprising administering one or more additional anticancer treatments or agents, wherein said method results in enhanced therapeutic efficacy relative to administration of the one or more checkpoint inhibitors, co-stimulatory checkpoint therapy, one or more additional anticancer treatments or agents, or non-viable, whole cell Mycobacterium alone.

In an embodiment or method of the invention, the one or more additional anticancer treatments or agents is selected from: adoptive cell therapy, surgical therapy, chemotherapy, radiation therapy, hormonal therapy, small molecule therapy such as metformin, receptor kinase inhibitor therapy, hyperthermia treatment, phototherapy, radioablation therapy, anti-angiogenic therapy, cytokine therapy, cryotherapy, biological therapy, HDAC inhibitor e.g. OKI-179, BRAF inhibitor, MEK inhibitor, EGFR inhibitor, VEGF inhibitor, P13K delta inhibitor, PARP inhibitor, mTOR inhibitor, hypomethylating agents, oncolytic virus, TLR agonist including TLR2, 3, 4, 5, 7, 8 or 9 agonists, such as MRx0518 (4D Pharma), STING agonists (including MIW815 and SYNB1891), and cancer vaccines such as GVAX or CIMAvax.

In another method of the invention, the anticancer treatment is selected from: microwave irradiation, radiofrequency ablation, targeted radiotherapy such as stereotactic ablative radiotherapy (SABR), embolisation, cryotherapy, ultrasound, high intensity focused ultrasound, cyberknife, hyperthermia, cryoablation, electrotome heating, hot water injection, alcohol injection, embolization, radiation exposure, photodynamic therapy, laser beam irradiation, and combinations thereof.

In an embodiment or method of the invention, the TLR agonists include mifamurtide (Mepact), Krestin (PSK), MRx0518 (4D Pharma), IMO-2125 (tilsotolimod), CMP-001, MGN-1703 (lefitolimod), entolimod, SD-101, GS-9620, imiquimod, resiquimod, MEDI4736, poly I:C, CPG7909, DSP-0509, VTX-2337 (motolimod), MEDI9197, NKTR-262, G100 or PF-3512676 and combinations thereof.

Suitable specific combinations include: Ipilimumab+MGN1703, Pembrolizumab+CMP001, Pembrolizumab+SD101, Tremelimumab+PF-3512676, resiquimod+pembolizumab.

In an embodiment or method of the invention, the chemotherapy comprises administration of one or more agents selected from: cyclophosphamide, methotrexate, 5-fluorouracil, doxorubicin, mustine, vincristine, procarbazine, prednisolone, bleomycin, vinblastine, dacarbazine, etoposide, cisplatin, epirubicin, capecitabine, leucovorin, folinic acid, carboplatin, oxaliplatin, gemcitabine, FOLFINROX, paclitaxel, pemetrexed, irinotecan and combinations thereof.

In an embodiment or method of the invention, the one or more additional anticancer treatments or agents is administered intratumorally, intraarterially, intravenously, intravascularly, intrapleuraly, intraperitoneally, intratracheally, intranasally, pulmonarily, intrathecally, intramuscularly, endoscopically, intralesionally, percutaneously, subcutaneously, regionally, stereotactically, orally or by direct injection or perfusion.

In an embodiment or method of the invention, the neoplasia, tumour or cancer is associated with a cancer selected from prostate cancer, liver cancer, renal cancer, lung cancer, breast cancer, colorectal cancer, breast cancer, pancreatic cancer, brain cancer, hepatocellular cancer, lymphoma, leukaemia, gastric cancer, cervical cancer, ovarian cancer, thyroid cancer, melanoma, carcinoma, head and neck cancer, skin cancer and soft tissue sarcoma, preferably wherein said neoplasia, tumour or cancer is associated with pancreatic, colorectal, prostate, skin or ovarian cancer, optionally wherein the neoplasia, tumour or cancer is metastatic.

In a further preferred embodiment or method of the invention, the neoplasia, tumour or cancer is associated with a sarcoma, preferably a soft tissue or non-soft tissue sarcoma. Particularly preferred non-soft tissue sarcomas include bone sarcomas (osteosarcoma, Ewing's sarcoma) and chondrosarcoma. Particularly preferred sarcomas include pleomorphic undifferentiated sarcoma (UPS), angiosarcoma, leiomyosarcoma, dedifferentiated liposarcoma (DDL), synovial sarcoma, rhabdomyosarcoma, epithelioid sarcoma, myxoid liposarcoma, alveolar soft part sarcoma, parachordoma/myoepithelioma, pleomorphic liposarcoma, extraskeletal myxoid chondrosarcoma, or malignant peripheral nerve sheath tumors. The patient may be less than 50 years of age, or less than 20 to 30 years of age, or a teenager or adolescent (<16 years of age), or a child (0 to 14 years of age). Optionally, the one or more sarcoma tumours demonstrate increased staining/expression of PD-L1 or PD-1. Optionally, the the non-viable whole cell Mycobacterium and/or checkpoint inhibitor and/or co-stimulatory binding agent is administered via intratumoral, peritumoral, perilesional or intralesional administration.

In an embodiment or method of the invention, the non-viable, whole cell Mycobacterium is selected from M. vaccae, M. obuense, M. parafortuitum, M. aurum, M. indicus pranii, M. phlei and combinations thereof, optionally in the form of a rough variant. Preferably, the the non-viable, whole cell Mycobacterium is M. obuense.

In an embodiment or method of the invention, the non-viable whole cell Mycobacterium and/or checkpoint inhibitor and/or co-stimulatory binding agent is administered via the parenteral, oral, sublingual, nasal or pulmonary route, preferably wherein said parenteral route is selected from subcutaneous, intradermal, subdermal, intraperitoneal, intravenous, intratumoral, peritumoral, perilesional or intralesional administration.

In an embodiment or method of the invention, the non-viable whole cell Mycobacterium is administered in an amount of from about 10⁴ to about 10¹⁰ cells, preferably about 10⁷ to about 10⁹ cells.

In further embodiments, methods of the invention include, one or more of the following: 1) reducing or inhibiting growth, proliferation, mobility or invasiveness of tumour or cancer cells that potentially or do develop metastases, 2) reducing or inhibiting formation or establishment of metastases arising from a primary tumour or cancer to one or more other sites, locations or regions distinct from the primary tumour or cancer; 3) reducing or inhibiting growth or proliferation of a metastasis at one or more other sites, locations or regions distinct from the primary tumour or cancer after a metastasis has formed or has been established, 4) reducing or inhibiting formation or establishment of additional metastasis after the metastasis has been formed or established, 5) prolonged overall survival, 6) prolonged progression free survival, 7) disease stabilisation, 8) increased quality of life.

In further embodiments, methods of the invention result in enhanced therapeutic efficacy as measured by a decrease or stabilisation of tumour size of one or more said tumours, optionally as defined by RECIST 1.1, including stable diseases (SD), a complete response (CR) or partial response (PR) of the target tumour; and/or stable disease (SD) or complete response (CR) of one or more non-target tumours.

In an embodiment or method of the invention, the checkpoint inhibitor refractory patient exhibits an innate (primary) resistance to checkpoint inhibitor therapy or an acquired (secondary) resistance to checkpoint inhibitor therapy, wherein (i) the patient exhibits an innate (primary) resistance to checkpoint inhibitor therapy as demonstrated by a lack of response or an insufficient response to said checkpoint inhibitor therapy, for at least about 8 weeks, or 12 weeks, or (ii) the patient exhibits an acquired (secondary) resistance to checkpoint inhibitor therapy as demonstrated by an initial response to said checkpoint therapy but with a subsequent relapse and progression of one or more tumours.

However, a therapeutic benefit or improvement need not be a cure or complete destruction of all target proliferating cells (e.g., neoplasia, tumour or cancer, or metastasis) or ablation of all pathologies, adverse symptoms or complications associated with or caused by cell proliferation or the cellular hyperproliferative disorder such as a neoplasia, tumour or cancer, or metastasis. For example, partial destruction of a tumour or cancer cell mass, or a stabilization of the tumour or cancer mass, size or cell numbers by inhibiting progression or worsening of the tumour or cancer, can reduce mortality and prolong lifespan even if only for a few days, weeks or months, even though a portion or the bulk of the tumour or cancer mass, size or cells remain.

Specific non-limiting examples of therapeutic benefit include a reduction in neoplasia, tumour or cancer, or metastasis volume (size or cell mass) or numbers of cells, inhibiting or preventing an increase in neoplasia, tumour or cancer volume (e.g., stabilizing), slowing or inhibiting neoplasia, tumour or cancer progression, worsening or metastasis, or inhibiting neoplasia, tumour or cancer proliferation, growth or metastasis.

In an embodiment of the invention, the combinations and methods disclosed herein provide a detectable or measurable improvement or overall response according to the irRC (as derived from time-point response assessments and based on tumour burden), including one of more of the following: (i) irCR—complete disappearance of all lesions, whether measurable or not, and no new lesions (confirmation by a repeat, consecutive assessment no less than 4 weeks from the date first documented), (ii) irPR—decrease in tumour burden ≥50% relative to baseline (confirmed by a consecutive assessment at least 4 weeks after first documentation).

An invention method may not take effect immediately. For example, treatment may be followed by an increase in the neoplasia, tumour or cancer cell numbers or mass, but over time eventual stabilization or reduction in tumour cell mass, size or numbers of cells in a given subject may subsequently occur.

In an embodiment of the invention, the combinations and methods disclosed herein result in a clinically relevant improvement in one or more markers of disease status and progression selected from one or more of the following: (i): overall survival, (ii): progression-free survival, (iii): overall response rate, (iv): reduction in metastatic disease, (v): circulating levels of tumour antigens such as carbohydrate antigen 19.9 (CA19.9) and carcinembryonic antigen (CEA) or others depending on tumour, (vii) nutritional status (weight, appetite, serum albumin), (viii): pain control or analgesic use, (ix): CRP/albumin ratio.

In a further embodiment, the checkpoint inhibition therapy comprises administration of a blocking agent, wherein said blocking agent is an antibody selected from the group consisting of: AMP-224 (Amplimmune, Inc), BMS-986016 or MGA-271, and combinations thereof. AMP-224, also known as B7-DCIg, is a PD-L2-Fc fusion soluble receptor described in WO2010/027827 and WO2011/066342.

BMS-986016 is a fully human antibody specific for human LAG-3 that was isolated from immunized transgenic mice expressing human immunoglobulin genes. It is expressed as an IgG4 isotype antibody that includes a stabilizing hinge mutation (S228P) for attenuated Fc receptor binding in order to reduce or eliminate the possibility of antibody- or complement-mediated target cell killing. The heavy and light chain amino acid sequences of BMS-986016 are provided in SEQ ID NOs: 17 and 18 of WO2015/042246.

In another embodiment, the checkpoint inhibition therapy comprises administration of BMS-986016 administered intravenously at a dose of between about 20 mg and about 8000 mg, every two weeks, optionally for a maximum of forty eight infusions.

In a further embodiment, the checkpoint inhibition therapy comprises administration of a blocking agent wherein said blocking agent is an antibody that specifically binds to B7-H3 such as enoblituzumab, an engineered Fc humanized IgG1 monoclonal antibody against B7-H3 with potent anti-tumor activity (Macrogenics, Inc.), or MGD009, a B7-H3 dual affinity re-targeting (DART) protein that bind both CD3 on T cells and B7-H3 on the target cell which has been found to recruit T cells to the tumor site and promote tumour eradication, or MGD009 is a humanized DART protein. MGC018 is anti-B7-H3 antibody drug conjugate (ADC) with a duocarmycin payload and cleavable peptide linker.

In some embodiments, the checkpoint inhibition therapy comprises administration of an anti-B7-H3-binding protein selected from the group consisting of DS-5573 (Daiichi Sankyo, Inc.), enoblituzumab (MacroGenics, Inc.), and omburtamab [8H9] (Y-mabs Therapeutics, Inc), an antibody against B7-H3 labeled with radioactive iodine (1-131).

In some embodiments, the checkpoint inhibition therapy comprises administration of indoleamine-2,3-dioxygenase (IDO) inhibitors such as D-I-methyl-tryptophan (Lunate) and other compounds described in U.S. Pat. No. 7,799,776, the contents of which are incorporated herein by reference.

In certain embodiments, the co-stimulatory checkpoint therapy upregulates the cellular immune system, wherein said co-stimulatory checkpoint therapy comprises administration of a binding agent, selected from a cell, protein, peptide, antibody or antigen binding fragment thereof, directed against CD27, OX40, GITR, or CD137, and combinations thereof, such as CD137 agonists including without limitation BMS-663513 (urelumab, an anti-CD137 humanized monoclonal antibody agonist, Bristol-Myers Squibb); agonists to CD40, such as CP-870,893 (a-CD40 humanized monoclonal antibody, Pfizer); OX40 (CD 134) agonists (e.g. anti-OX40 humanized monoclonal antibodies, AgonOx and those described in U.S. Pat. No. 7,959,925), and Astra Zeneca's MEDI0562, a humanised OX40 agonist; MEDI6469, murine OX4 agonist; and MEDI6383, an OX40 agonist; or agonists to CD27 such as CDX-1127 (a-CD27 humanized monoclonal antibody, Celldex). Suitable anti-GITR antibodies include TRX518 (Tolerx), MK-1248 (Merck), CK-302 and suitable anti-4-1BB antibodies for use in the invention include PF-5082566 (Pfizer).

TIGIT is a checkpoint receptor thought to be involved in mediating T cell exhaustion in tumours; It has been shown that TIGIT, but not the other checkpoint molecules CTLA-4 and PD-1, was associated with NK cell exhaustion in tumour-bearing mice and patients with colon cancer. Blockade of TIGIT prevents NK cell exhaustion and promoted NK cell-dependent tumour immunity in several tumour-bearing mouse models. Furthermore, blockade of TIGIT results in potent tumour-specific T cell immunity in an NK cell-dependent manner, enhanced therapy with antibody to the PD-1 ligand PD-L1 and sustained memory immunity in tumour re-challenge models.

Evidence suggests that greater infiltration of MDSCs, including CD68+ or CD163+ specific tumour-associated macrophages (TAMs), correlates with checkpoint inhibitor resistance. In vivo studies have also demonstrated that suppression of CD103+ DCs recruitment by β-catenin signalling results in primary resistance.

The loss of β2 microglobulin (B2M) is one mechanism of acquired resistance to immunotherapy which results from defective antigen presentation. B2M loss interferes with MHC class I heavy chain folding, leading to a loss of its receptor localization and interruption of downstream signalling, which would otherwise propagate T-cell activation and recruitment. Tumour downregulation of MHC class I molecules is an alternative mechanism of tumour immune escape which renders antitumour T-cell responses ineffective

The function of APCs in antitumor immunity is to transfer tumour antigens to tumour-draining lymph nodes for tumor-specific CD8+ T-cell priming. In melanoma, CD103+ DCs are the only APCs that have such a function. In melanoma mouse models, administration of the growth factor FLT3L and poly I:C has expanded and activated CD103+DC progenitors in the tumour, thereby reversing anti-PD-L1 resistance. Furthermore, studies show that failed accumulation of CD103+ dendritic cells, a cell type that is the major source of the T cell-recruiting chemokines CXCL9/10, in non-inflamed tumours mediates deficient entry of therapeutically activated T cells and immunotherapy resistance. Therefore, absence of CD103+ DCs from the tumour microenvironment may be a dominant mechanism of resistance to multiple immunotherapies.

Conventional dendritic cells (cDCs) are specialized antigen-presenting cells that control T cell immunity. Lineage tracing experiments in mice have mapped two developmentally and functionally distinct populations, cDC1 and cDC2, that reside in peripheral tissues where they are defined by expression of CD103 and CD11 b, respectively. These lineages and their functions are conserved in humans. Of these, cDC1 are highly efficient at cross-presenting antigens to cytotoxic T cells and are the major stimulatory cDC population within tumours, both for the generation of anti-tumor immunity in draining lymph nodes (LNs) and upon direct interaction with effector T cells within the tumor microenvironment. Furthermore, cDC1 are critical for therapeutic responses to checkpoint blockade

TIM-3 is highly expressed by intratumoral CD103+ dendritic cells and administration of a TIM-3 antibody indirectly enhances a CD8+ T cell response during chemotherapy.

The TLR3 agonist polyriboinosinic:polyribocytidylic acid (poly I:C), induces type I IFN production as well as DC maturation. CD141+DC are the human equivalents of murine CD8+/CD103+DC and TLR3 and TLR8 are expressed by CD141+DC. Injection of mice with TLR3 and TLR7 agonists (resiquimod) results in upregulation of costimulatory molecules CD80, CD83 and CD86 by CD141+ and CD1c+DC alike.

The term “combination” as used throughout the specification, is meant to encompass the administration of the checkpoint inhibitor and/or co-stimulatory checkpoint binding agent simultaneously, separately or sequentially with administration of the Mycobacterium. Accordingly, the checkpoint inhibitor and/or co-stimulatory checkpoint binding agent and the Mycobacterium may be present in the same or separate pharmaceutical formulations, and administered at the same time or at different times.

Thus, a non-viable whole-cell Mycobacterium and the checkpoint inhibitor and/or co-stimulatory checkpoint binding agent may be provided as separate medicaments for administration at the same time or at different times.

Preferably, a non-viable whole-cell Mycobacterium and checkpoint inhibitor and/or co-stimulatory checkpoint binding agent are provided as separate medicaments for administration at different times. When administered separately and at different times, either the non-viable whole-cell Mycobacterium or checkpoint inhibitor and/or co-stimulatory checkpoint binding agent may be administered first; however, it is suitable to administer checkpoint inhibitor and/or co-stimulatory checkpoint binding agent followed by the non-viable whole-cell Mycobacterium. In addition, both can be administered on the same day or at different days, and they can be administered using the same schedule or at different schedules during the treatment cycle.

In an embodiment of the invention, a treatment cycle consists of the administration of a non-viable whole-cell Mycobacterium daily, weekly fortnightly or monthly, simultaneously with checkpoint inhibitor and/or co-stimulatory checkpoint binding agent weekly, or every two weeks or every three weeks or every four weeks or more. Alternatively, the non-viable whole-cell Mycobacterium is administered before and/or after the administration of the checkpoint inhibitor and/or co-stimulatory checkpoint binding agent.

In another embodiment of the invention, the non-viable whole-cell Mycobacterium is administered to the patient before and after administration of a checkpoint inhibitor and/or co-stimulatory checkpoint binding agent. That is, in one embodiment, the whole cell, non-pathogenic heat-killed Mycobacterium is administered to the patient before and after said checkpoint inhibitor and/or co-stimulatory checkpoint binding agent.

In another embodiment of the invention, the non-viable whole-cell Mycobacterium is administered to the patient before and after administration of a checkpoint inhibitor and/or co-stimulatory checkpoint binding agent and/or one or more additional anticancer treatments or agents, which include: adoptive cell therapy, surgical therapy, chemotherapy, radiation therapy, hormonal therapy, small molecule therapy such as metformin, receptor kinase inhibitor therapy, hyperthermia treatment, phototherapy, radioablation therapy, anti-angiogenic therapy, cytokine therapy, cryotherapy, biological therapy, HDAC inhibitor e.g. OKI-179, BRAF inhibitor, MEK inhibitor, EGFR inhibitor, VEGF inhibitor, P13K delta inhibitor, PARP inhibitor, mTOR inhibitor, hypomethylating agents, oncolytic virus, TLR agonist including TLR2, 3, 4, 5, 7, 8 or 9 agonists, such as MRx0518 (4D Pharma), STING agonists (including MIW815 and SYNB1891), and cancer vaccines such as GVAX or CIMAvax.

Dose delays and/or dose reductions and schedule adjustments are performed as needed depending on individual patient tolerance to treatments.

Alternatively, the administration of checkpoint inhibitor and/or co-stimulatory checkpoint binding agent may be performed simultaneously with the administration of the effective amounts of non-viable whole-cell Mycobacterium.

The subject whom is to undergo checkpoint inhibition therapy and/or co-stimulatory checkpoint therapy according to the present invention may do so simultaneously, separately or sequentially with administration of the non-viable whole-cell Mycobacterium.

In an aspect of the invention, the effective amount of the non-viable whole-cell Mycobacterium may be administered as a single dose. Alternatively, the effective amount of the non-viable whole-cell Mycobacterium may be administered in multiple (repeat) doses, for example two or more, three or more, four or more, five or more, ten or more, or twenty or more repeat doses. Wherein multiple doses of Mycobacterium are administered there may be a time period of 1 week, 2 weeks, 3 weeks, 4 weeks or a combination of the aforementioned between doses.

The non-viable whole-cell Mycobacterium may be administered between about 8 weeks, 6 weeks or 4 weeks and/or about 1 day prior to checkpoint inhibition therapy, such as between about 4 weeks and 1 week, or about between 3 weeks and 1 week, or about between 3 weeks and 2 weeks. Administration may be presented in single or more preferably, in multiple doses.

In one embodiment of the present invention, the non-viable whole-cell Mycobacterium may be in the form of a medicament administered to the patient in a dosage form.

A container according to the invention in certain instances, may be a vial, an ampoule, a syringe, capsule, tablet or a tube. In some cases, the mycobacteria may be lyophilized and formulated for resuspension prior to administration.

However, in other cases, the mycobacteria are suspended in a volume of a pharmaceutically acceptable liquid. In some of the most preferred embodiments there is provided a container comprising a single unit dose of mycobacteria suspended in pharmaceutically acceptable carrier wherein the unit dose comprises about 1×10³ to about 1×10¹² organisms, or about 1×10⁶ to about 1×10⁹ organisms. In some very specific embodiments the liquid comprising suspended mycobacteria is provided in a volume of between about 0.01 ml and 10 ml, or between about 0.03 ml and 2 ml or between about 0.1 ml and 1 ml. The foregoing compositions provide ideal units for immunotherapeutic applications described herein.

Embodiments discussed in the context of a methods and/or composition of the invention may be employed with respect to any other method or composition described herein. Thus, an embodiment pertaining to one method or composition may be applied to other methods and compositions of the invention as well.

In some cases, the non-viable whole-cell Mycobacterium are administered to specific sites on or in a subject. For example, the mycobacterial compositions according to the invention, such as those comprising M. obuense in particular, may be administered into or adjacent to tumours or adjacent to lymph nodes, such as those that drain tissue surrounding a tumour. Thus, in certain instances sites administration of mycobacterial composition may be near the posterior cervical, tonsillar, axillary, inguinal, anterior cervical, sub-mandibular, sub mental or superclavicular lymph nodes.

The non-viable whole-cell Mycobacterium may be administered for the length of time the cancer or tumour(s) is present in a patient or until such time the cancer has regressed or stabilized. The whole cell, non-pathogenic heat-killed Mycobacterium may also be continued to be administered to the patients once the cancer or tumour has regressed or stabilised.

Mycobacterial compositions according to the invention will comprise an effective amount of mycobacteria typically dispersed in a pharmaceutically acceptable carrier. The phrases “pharmaceutically or pharmacologically acceptable” refers to molecular entities and compositions that do not produce an adverse, allergic or other untoward reaction when administered to an animal, such as, for example, a human, as appropriate. The preparation of an pharmaceutical composition that contains mycobacteria will be known to those of skill in the art in light of the present disclosure, as exemplified by Remington's Pharmaceutical Sciences, 18th Ed. Mack Printing Company, 1990, Moreover, for animal (e.g., human) administration, it will be understood that preparations should meet sterility, pyrogenicity, general safety and purity standards. A specific example of a pharmacologically acceptable carrier as described herein is borate buffer or sterile saline solution (0.9% NaCl).

As used herein, “pharmaceutically acceptable carrier” includes any and all solvents, dispersion media, coatings, surfactants, antioxidants, preservatives {e.g., antibacterial agents, antifungal agents), isotonic agents, absorption delaying agents, salts, preservatives, drugs, drug stabilizers, gels, binders, excipients, disintegration agents, lubricants, sweetening agents, flavouring agents, dyes, such like materials and combinations thereof, as would be known to one of ordinary skill in the art (see, for example, Remington's Pharmaceutical Sciences, 18th Ed. Mack Printing Company, 1990, pp. 1289-1329).

In a preferred embodiment, the non-viable whole-cell Mycobacterium is administered via a parenteral route selected from subcutaneous, intradermal, subdermal, intraperitoneal, intravenous and intravesicular injection, or intratumoral, peritumoral, perilesional or intralesional administration.

Intradermal injection enables delivery of an entire proportion of the mycobacterial composition to a layer of the dermis that is accessible to immune surveillance and thus capable of electing an anti-cancer immune response and promoting immune cell proliferation at local lymph nodes.

Though in highly preferred embodiments of the invention mycobacterial compositions are administered by direct intradermal injection, it is also contemplated that other methods of administration may be used in some case. Thus in certain instances, the non-viable whole-cell Mycobacterium of the present invention can be administered by injection, infusion, continuous infusion, intravenously, intradermally, intraarterially, intraperitoneally, intralesionally, intravitreally, intravaginally, intrarectally, topically, intratumourally, intramuscularly, intraperitoneally, subcutaneously, subconjunctival, intravesicularlly, mucosally, intrapericardially, intraumbilically, intraocularally, orally, intracranially, intraarticularly, intraprostaticaly, intrapleurally, intratracheally, intranasally, topically, locally, inhalation (e.g. aerosol inhalation), via a catheter, via a lavage, or by other method or any combination of the forgoing as would be known to one of ordinary skill in the art (see, for example, Remington's Pharmaceutical Sciences, 18th Ed. Mack Printing Company, 1990).

In another embodiment, the immunomodulator is to be administered into the skin of a checkpoint inhibitor refractory patient via a microneedle device comprising a plurality of microneedles.

Table 1 below presents various methodologies and formulation approaches for fabricating solid microneedles according to the invention.

| Methods | Materials | Dimensions (μm) |
|---|---|---|
| Separable dissolving Arrow Heads | (polydimethylsiloxane (PDMS) | 600 μm-long PVP/PVA |
| | Sylgard, | arrowhead capped onto |
| | Metal shaft | a metal shaft with an |
| | Water soluble excipients-PVP, sucrose | exposed length of 600 |
| | PLGA | μm and a 100 μm |
| | | overlap. |
| Dissolving | Mixture Inulin, | basal diameter: |
| | water, dextrin | 3.24 ± 0.16 and 0.55 ± 0.03 |
| | | mm |
| Pyramidal dissolving Polymer | polyvinyl alcohol (PVA), Polyvinylpyrrolid | base width × base depth × |
| | one (PVP) | needle height: |
| | A = Pyramidal MN | 300 μm × 300 μm × 600 |
| | B = Extended pyramidal MNs | μm. |
| | C = Pedestal MNs | 300 μm × 300 μm × 900 |
| | | μm. |
| | | 340 μm × 340 μm × 900 |
| | | μm) |
| | | 10 × 10 array |
| Deep reactive Ion etching | Chromium | 120 μm length, <1 μm tip |
| | Silicon wafers | diameter |
| Dissolving. Fabricated by a drawing | Maltose | 1200 μm length |
| technique to create a sharp tip | | 60 μm tip diameter |
| Cutting metal using infrared laser, | Metal | 1000 μm length, |
| manually bending the MN structure, | | 50 μm × 200 μm cross |
| electropolished | | section at base, tapered |
| | | to a sharp tip (angle 20°), |
| | | 106 array |
| Cutting metal using infrared laser, | Metal | 1000 μm length, |
| manually bending the MN structure, | | 50 μm × 200 μm cross |
| electropolished | | section at base, tapered |
| | | to a sharp tip (angle 20°), |
| | | 106 array |
| Silicon master mould to make | Sugar glass MN: | 200 μm |
| PDMS inverse mould | Trehalose/mannitol (50:50 w/w) | base 20 |
| | Trehalose dehydrate/sucrose | μm tip |
| | (75:25 w/w) | 300 μm height |
| | Trehalose/sucrose (75:25 w/w) | |
| | Trehalose/sucrose (50:50 w/w) | |
| Stainless steel MN produced by | Stainless steel | 750 pm height |
| chemical etching | | 200 pm × 50 pm at the |
| | | base. Single row of 5 |
| | | MNs |
| Dissolving MN | Starch/gelatin (1:1 ratio) | 600 pm |
| | PDMS mould | height 300 |
| | | pm base 5 |
| | | pm tip |

Table 2 below presents a selection of microneedle device technologies for use according to the invention, said patents and patent application herein incorporated by reference.

| Patent Number | Date of Filing | Applicant |
|---|---|---|
| U.S. 2010042050 | 16^{th} Apr. 2007 | Nemaura Pharma Ltd, USA |
| WO 2011016230 | 4^{th} Aug. 2010 | Medrx Co., Ltd., Japan |
| WO 2011084951, | 4^{th} Jan. 2011, | Ratio, Inc., |
| U.S. 20110172645 | 8^{th} Jan. 2010 | USA |
| WO 2011014514 | 27^{th} Jul. 2010 | 3M Innovative Properties |
| CA 2696810 | 15^{th} Jan. 2010 | Bioserentach |
| JP 2011078618 | | Co. Ltd., |
| WO 2011043086 | | Japan |
| AU 2010201434 | | |
| KR 2011067009 | | |
| EP 2343102 A1 | | |
| WO 2013096026 | 12^{th} Dec. 2012 | 3M Innovative Properties |
| WO 2013066262 | 2^{nd} Nov. 2011 | Singapore |
| EP 2289843 | 31^{st} Aug. 2009 | University College Cork-National |
| | | University of Ireland, |
| | | Cork, Ireland. |
| JP 2011083387 | 14^{th} Oct. 2009 | Kyushu Institute of Technology, |
| | | Japan; Nichiba n Co., Ltd. |
| CN 102000020 | 17^{th} Nov. 2010 | Beijing Pharmaceutical Research |
| | | Institute, Henan Lingrui |
| | | Pharmaceutical Co., Ltd., |
| CN 103263727 | 22^{nd} May 2013 | Tsinghua University, People |
| | | Republic of China. |
| U.S. 20130030374 | 11 Oct. 2012 | Toppan Printing Co., Ltd. |
| CN 103181887 | 30 Dec. 2011 | Shanghai No. 7 People's |
| | | Hospital, People |
| | | Republic of China |
| GB2472778A | 17^{th} Aug. 2009 | PANGAEA LAB LTD |
| WO 2011026144 or | 31^{st} Aug. 2009 | AllTranz Inc., USA |
| 20110052694 | | |
| U.S. 20110118560, | 13^{th} Nov. 2009. | Searete LLC, USA |
| 20110118656, U.S. | 19^{th} Feb. 2010, | |
| 20110117150, U.S. | 9ₜₕ Mar. 2010 | |
| 20110118652, U.S. | | |
| CN 101991846 | 17^{th} Nov. 2010 | Chifeng Boen Pharmaceutical Co., |
| | | Ltd., People Republic of China |
| U.S. 20130171722 | 3^{rd} Jan. 2012 | City University of Hong |
| | | Kong, Hong Kong |

Other preferred microneedle devices for use according to the invention include: North Carolina State University (as described in WO2017/151727), Debioject microneedle (Debiotech, Switzerland), Micronject600 (NaoPass, Israel, as described in WO2008/047359), Nanopatch (Vaxxas, USA), SOFUSA (Kimberly-Clark, USA, as described in WO2017/189259 and WO2017/189258), Micron Biomedical's dissolving microarray, and the MIMIX dissolving, controlled release microarray (Vaxess, USA).

In an embodiment, the present invention provides an immunomodulator for use in the treatment, reduction, inhibition or control of cancer in a subject, wherein the immunomodulator comprises a whole cell, non-viable Mycobacterium and wherein said immunomodulator is to be administered into the skin of said subject via a microneedle device comprising a plurality of microneedles.

In an embodiment, the microneedles are hollow. In a separate embodiment the microneedles are solid.

In a further embodiment, the plurality of microneedles are deployed in a line, square, circle, grid or array.

In a further embodiment, the microneedle device includes between 2 and 2000 microneedles per square centimetre, such as between 4 and 1500 microneedles per square centimetre, or between 10 and 1000 microneedles per square centimetre.

In a further embodiment, the microneedles are between 2 and 2000 microns in length, such as between 20 and 1000 microns, or between 50 and 500 microns, or between 100 and 400 microns.

In a further embodiment, the microneedles are configured to deliver the immunomodulator intradermally, optionally wherein said immunomodulator is delivered to the lymphatic vessels.

In a further embodiment, the said immunomodulator is coated onto or embedded within at least a portion of the microneedles, optionally wherein the microneedles are implanted into or removable from the skin. Preferably, said coating or microneedle is dissolvable upon contact with the skin.

In a further embodiment, wherein said microneedles are hollow and said immunomodulator is delivered intradermally as a suspension through said microneedles, optionally wherein said microneedles are implanted into or removable from the skin.

In a further embodiment, there is provided a method of treating, reducing, inhibiting or controlling a neoplasia, tumour or cancer in a checkpoint inhibitor refractory patient, wherein said method comprises:

(i) providing a microneedle device comprising a plurality of microneedles,
(ii) causing the microneedles to penetrate into the skin of the subject and assume an anchored state in which the microneedles are anchored in the skin and project from the microneedle device,
(iii) delivering into the skin via the microneedle a quantity of an immunomodulator, wherein said immunomodulator comprises a whole cell, non-viable Mycobacterium.

In a further embodiment, there is provided a kit of parts for delivering at least on immunomodulator into the skin of a checkpoint inhibitor refractory patient, comprising:

a microneedle device comprising a plurality of microneedles, and, one or more immunodulators selected from:
a whole cell non-viable Mycobacterium such as M. vaccae, such as NCTC 11569, M. obuense, such as NCTC 13365, M. parafortuitum, M. aurum, M. indicus pranii, M. phlei and combinations thereof, and;
a checkpoint inhibitor, selected from a cell, protein, peptide, antibody or antigen binding fragment thereof, directed against CTLA-4, PD-1, PD-L1, PD-L2, LAG-3, B7-H3, B7-H4, B7-H6, A2AR, or IDO, and combinations thereof.

In a further embodiment, there is provided a microneedle device comprising a plurality of microneedles, and contained thereon or therein a composition comprising a whole cell, non-viable Mycobacterium.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and immunology or related fields are intended to be within the scope of the following claims.

The invention is further described with reference to the following non-limiting Examples.

### Example 1

Adult C57BL/6 mice were injected subcutaneously on the flank with 10⁵ cells from a pancreatic cancer cell line obtained from KPC mice (Hingorani et al. Cancer Cell, 2005, 7:469-48). These murine pancreatic cancer cells bear mutations in Kras, p53 and Pdx-Cre (Hingorani et al. Cancer Cell, 2005, 7:469-48).

When the injected tumour cells had grown to become a palpable tumour (day 0), mice were left untreated or received treatment with:
1) 0.1 mg heat-killed whole cell M. obuense NTCT 13365/mouse, subcutaneously alternating injections in the scruff of the neck with those at the base of the tail on alternating days over 5 day period with a 2 day break for the length of the study;
2) 10 mg/kg anti-PDL-1 mAb intraperitoneally once weekly;
3) the combination of anti-PDL-1 and M. obuense NTCT 13365 at a dose and schedule described above for the two compounds used singly.

Tumour growth was monitored over the course of the study to determine whether the treatment administered had an effect on reducing tumour size and improving prospects of survival.

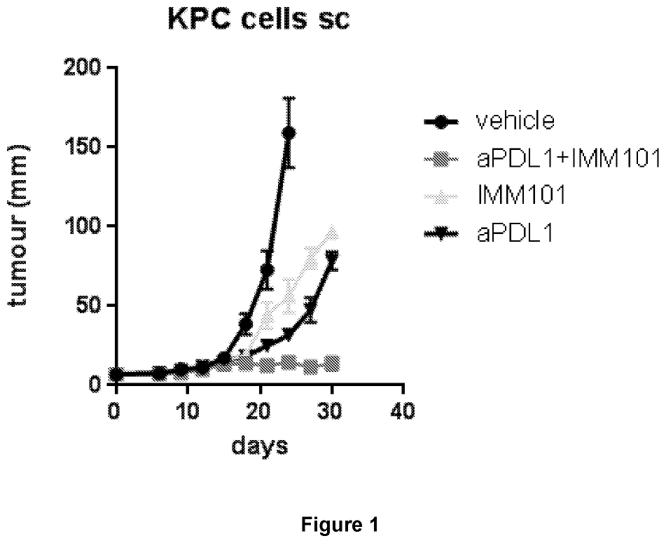

Data presented in FIG. 1 show that mice which received the treatment combination of anti-PDL-1 and M. obuense NTCT 13365 demonstrated a continued reduction in tumour size and appeared to control the tumour. This reduction in tumour size was more pronounced compared to mice receiving either treatment alone. Mice left untreated had uncontrolled tumour growth and soon succumbed to the disease.

### Example 2

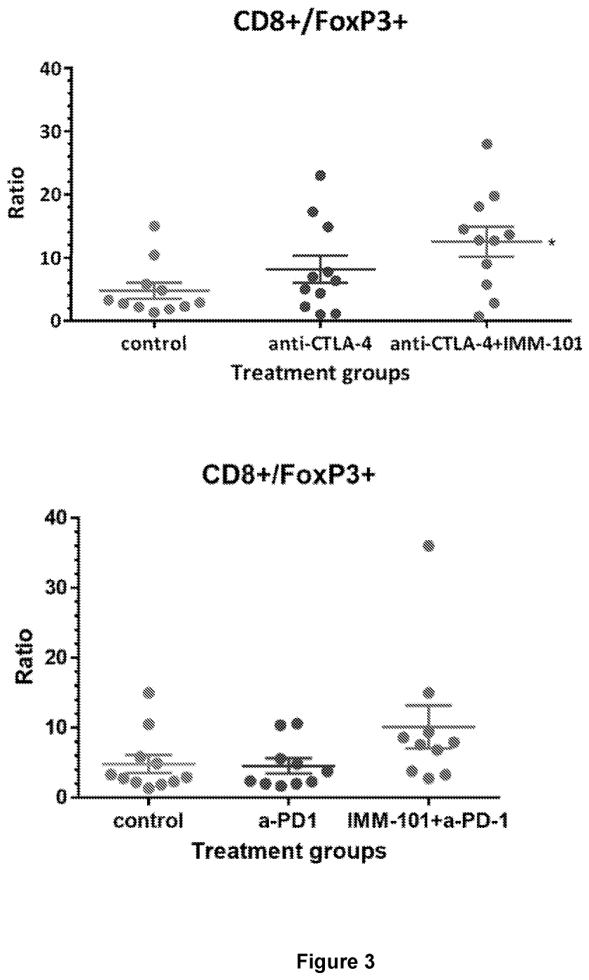

The effects of combination treatment with IMM-101 (heat-killed whole cell M. obuense NTCT 13365) and checkpoint inhibitors were investigated in C57BL/6 mice bearing subcutaneous checkpoint resistant B16-F10 tumours. The mice were engrafted at D0. Mice were randomized on D1 and received a total of 8 SC injections of IMM-101 at 0.1 mg/mouse on D1, D3, D5, D7, D9, D11, D13 and D15 (half of surviving mice) or D16 (half of surviving mice) (Q2D×8) or a total of 4 IP injections of anti-PD1 or anti-CTLA4 at 10 mg/kg (twice weekly for two consecutive weeks on D1, D5, D8 and D12: TW×2) alone or in combination. At Day 15 and 16, half of all surviving mice were terminated after the last treatment and whereupon tumour immune infiltrate cells and spleen immune cells (the ratio of CD8+ cells and FoxP3 Treg cells) characterization were quantified by FACS analysis (FIG. 3). As can be seen, there is an enhanced ratio of CD8+ to Tregs which would translate to enhanced efficacy in tumour regression in human checkpoint refractory patients.

### Example 3

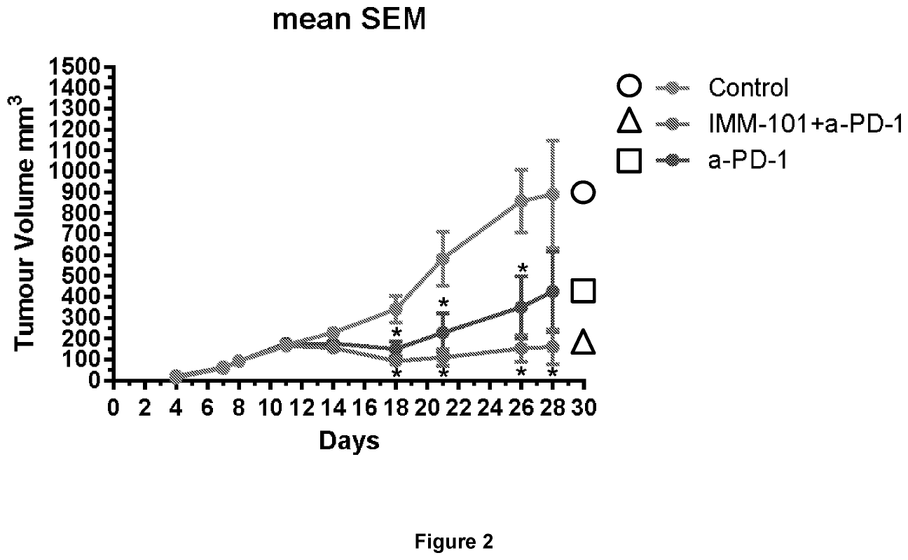

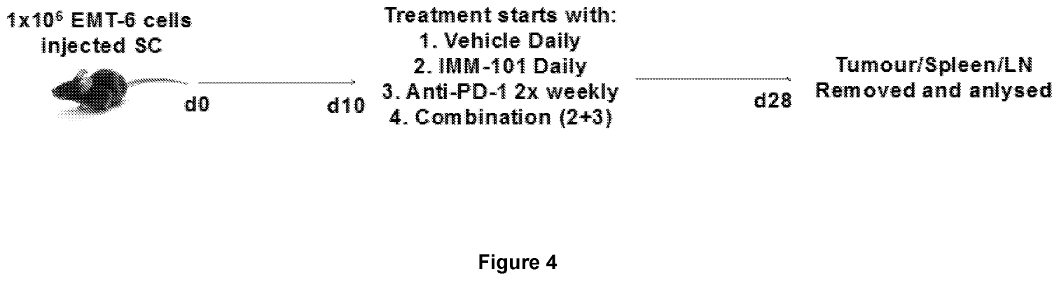

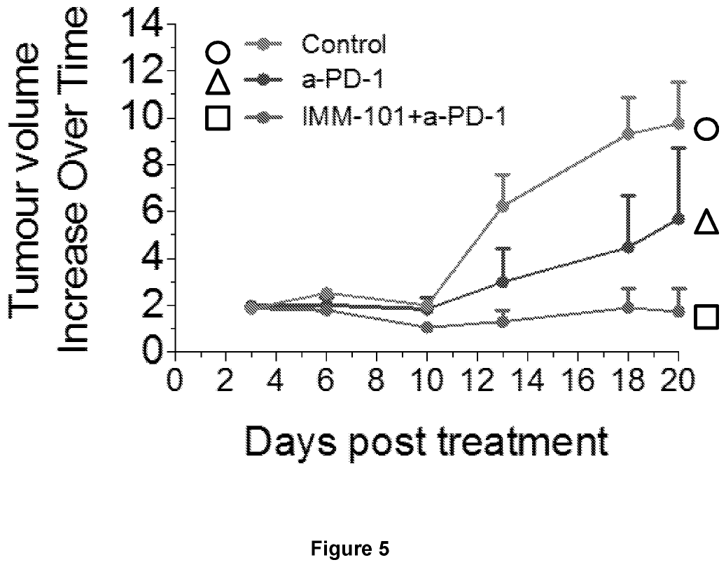

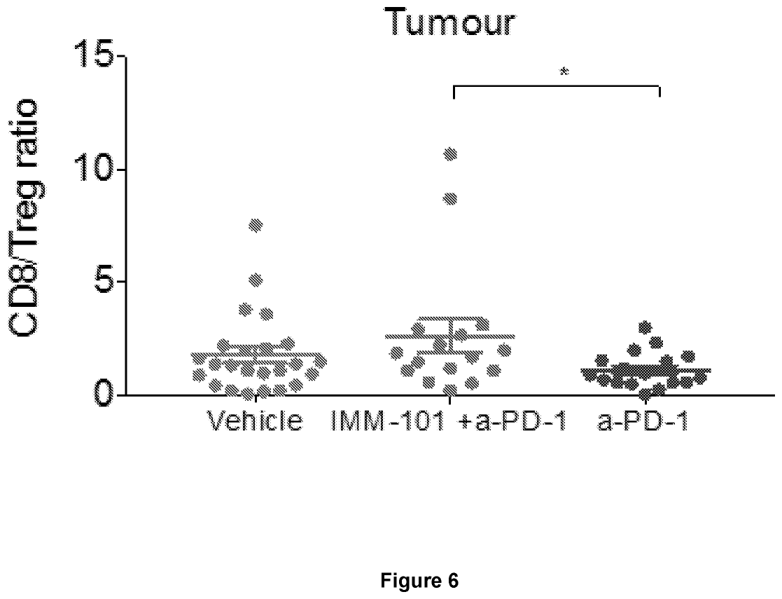

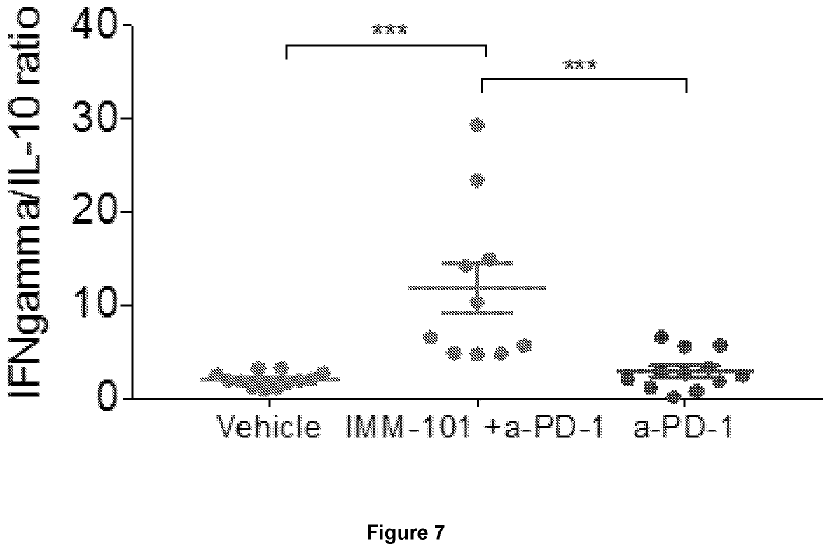

BALB/C mice were injected s.c. with 1×10⁶ EMT-6 mouse mammary tumour cells. At the day of tumour mean volume reaching 80-120 mm² treatment commenced (around day 7), with 0.1 mg/mouse IMM-101 daily, 10 mg/kg/injection of anti-PD-1 twice weekly, a combination of IMM-101 and anti-PD-1 or vehicle (FIG. 4). At day 28, mice were euthanised and tumour draining lymph node and spleen were removed from all mice. Mouse tumour volume was measured every 3 days. Doubling time (ratio of tumour size from size at treatment commencement) of tumour size post treatment was measured (FIG. 5) and tumour volume plotted against time (FIG. 2). The ratio of CD8⁺ T cells/FoxP3⁺ regulatory T cells at day 28 was measured by flow cytometry (combination of 2 experiments) (FIG. 6), and the ratio of IFN-γ/IL-10 measured by ELISA in the supernatant of spleen cells stimulated with anti-CD3 at day 28 for 72 hours (FIG. 7). (* p<0.05, ** p<0.01, *** p<0.001). As can be seen, there is an enhanced ratio of CD8+ to Tregs and an increased ratio of IFN-γ/IL-10 which would translate to enhanced efficacy in tumour regression in human patients.

### Example 4

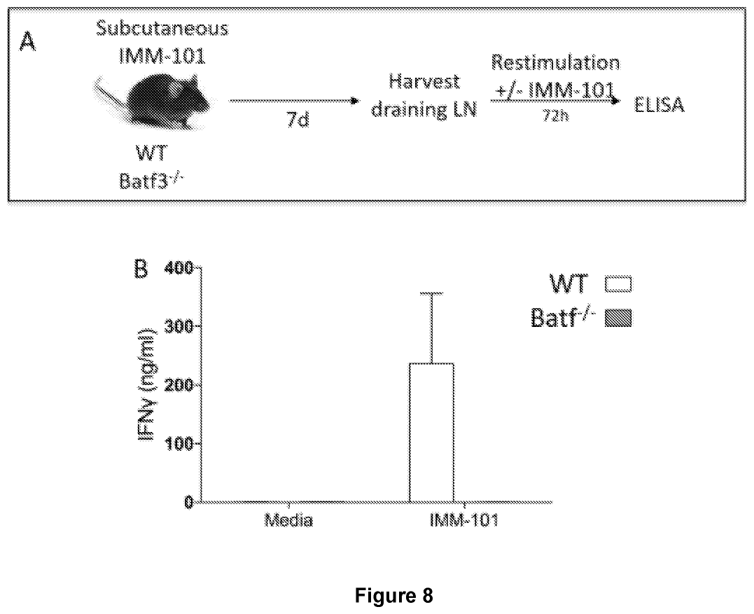

C57BL/6 VVT or Batf3−/− mice were injected s.c. in the footpad with IMM-101 (300 μg). Draining LNs were harvested 7 days later and restimulated for 72 hours with IMM-101 or media alone. A) Schematic of experimental procedure B) IFN-g levels were measured by ELISA in the supernatants following restimulation. The ability of IMM-101 to induce IFN-gamma secretion in vivo is lost in Baft3−/− mice suggesting a requirement for CD103+ dendritic cells in this pathway (FIG. 8).

### Example 5

A study has been developed to investigate the effects of a preparation of heat-killed whole cell M. obuense (IMM-101) in combination with A Study of the Safety and Efficacy of IMM-101 in Combination with Checkpoint Inhibitor Therapy in Patients with Advanced Melanoma radiation-induced immunogenic tumour necrosis in patients with previously treated colorectal cancer.

Patients to be treated exhibit unresectable, Stage III or Stage IV metastatic melanoma who are either previously untreated (cohort A), or whose disease has progressed during PD-1 blockade (cohort B).

This study seeks to investigate whether the combination of IMM-101 with nivolumab is well-tolerated and to investigate efficacy signals of the combination, both in treatment-naive patients (cohort A) and in those whose disease has progressed during PD-1 blockade (cohort B—checkpoint refractory/resistant patients).

IMM-101 is administered as a single 0.1 mL intradermal injection of IMM-101 (10 mg/mL) into the skin overlying the deltoid muscle, with the arm being alternated between each dose. The Investigator will have been appropriately trained a priori in the technique of intradermal injection.

Previous clinical experience with IMM-101 has suggested that this dose is safe and well tolerated. The skin reaction that develops at the site of injection is characterised by erythema, local swelling and occasionally mild ulceration. All symptoms are to be expected given the known pharmacology of the product and previous clinical experience. Furthermore, data from safety and tolerability studies with IMM-101 have revealed that skin reactions resolve satisfactorily over time and do not impair daily activity.

The first dose of IMM-101 administered to each patient in the study is followed by vital signs monitoring for at least 2 hours under medical supervision with resuscitation facilities available as a precautionary measure.

The treatment regimen will be 1 dose of IMM-101 given every 2 weeks for the first 3 doses followed by a rest period of 4 weeks, then one dose every 2 weeks for the next 3 doses. This will be followed by a dose every 4 weeks thereafter with a window of +/−2 days allowed.

Nivolumab and ipilimumab are administered according to the prescribing information. When either nivolumab or ipilimumab are administered on the same day as IMM-101, according to the Schedule of Assessments, patients will receive IMM-101 first. The first dose of nivolumab administered to each patient on study is given at least 2 hours after the first dose of IMM-101.

Ipilimumab may be used as a subsequent treatment in place of nivolumab alongside IMM-101 for patients in cohort B either because they continue to progress on study according to RECIST 1.1 and/or investigator decision that continuing to receive nivolumab is no longer appropriate due to clinical progression.

Treatment for patients in both cohorts is continued until disease progression (as assessed by Response Evaluation Criteria in Solid Tumours [RECIST] 1.1) subject to the following qualifications: unacceptable side-effects, the investigator's decision to discontinue treatment, withdrawal of patient consent, or 18 months of IMM-101 treatment, whichever is the sooner. Patients with a complete response maintained over 2 scans should continue treatment unless the investigator considered this not in the patient's best interest. Patients in cohorts A and B who have documented disease progression may continue treatment with nivolumab+IMM-101 on study if they have a clinical benefit and no decline in performance status, no clinically relevant adverse effects with the study treatment as determined by the investigator, or are not deemed to require alternative treatment.

Patients in cohort B who fail to respond to treatment with IMM-101+nivolumab i.e., they have either documented progression by RECIST 1.1, or clinical progression (but without meeting the RECIST 1.1 rules for progressive disease), and, in both cases have no prior recorded response, have the option to change treatment on study to ipilimumab+IMM-101 if the investigator considers this in the patient's best interest and the patient has not previously received ipilimumab off study (monotherapy or in combination). This treatment may continue until the maximum 4 doses of ipilimumab have been received or stop sooner due to unacceptable side-effects, the investigator's decision to discontinue treatment, withdrawal of patient consent or 18 months of IMM-101 treatment, whichever is the sooner. Patients in cohort B who receive all 4 doses of ipilimumab should remain on study after this time and follow the protocol assessments. They may continue to receive IMM-101 during this period until unacceptable side-effects, the investigator's decision to discontinue treatment, withdrawal of patient consent or 18 months of IMM-101 treatment, whichever is the sooner. Following baseline assessments, all patients will be followed up for assessment of safety, response to treatment (via scheduled scans) and survival, according to the Study Schedule of Assessments with all patients allowed the opportunity of 18 months of IMM-101 treatment on study. The first post-baseline scheduled scan is at week 12 for patients in cohort A and at week 6 for those in cohort B. Subsequent scans are every 8 weeks with unscheduled scans allowed if clinically indicated, for example to confirm progression. At the discretion of the investigator, the frequency of scans may be increased to every 12 weeks for patients who continue on study beyond week 52.

Nivolumab will be administered as 3 mg/kg IV infusion every two weeks in accordance with the prescribing information. In instances when nivolumab and IMM-101 are given on the same day, IMM-101 will be administered first. The first dose of nivolumab administered to each patient on study is given at least 2 hours after the first dose of IMM-101. In the event of toxicity, doses may be delayed.

If used on study for patients in cohort B, ipilimumab will be administered as a 3 mg/kg IV infusion over 90 minutes every three weeks for a maximum of 4 doses, in accordance with the prescribing information. The first dose of ipilimumab can start at any time during the study but must be at least 2 weeks after the last dose of nivolumab. In instances when ipilimumab and IMM-101 are given on the same day, IMM-101 will be administered first. In the event of toxicity, doses may be delayed, but all ipilimumab doses must be administered within 16 weeks of the first dose.

Patients in cohort B who receive all 4 doses of ipilimumab should remain on study after this time and follow the protocol assessments. They may continue to receive IMM-101 during this period until unacceptable side-effects, the Investigator's decision to discontinue treatment, withdrawal of patient consent or 18 months of IMM-101 treatment, whichever is the sooner.

### Example 6

The effects of combination treatment with IMM-101 (heat-killed whole cell M. obuense NTCT 13365) and a checkpoint inhibitor were investigated in C57BL/6j mice bearing subcutaneous checkpoint resistant B16-F10 (melanoma) tumours, as used in Example 2. The mice were inoculated with 50,000 tumour cells and then randomized with 10 mice per group when tumour volumes reached between 54 and 125 mm³ (mean TV ranged from 82 to 88 mm³ across the groups). Animals were dosed on Day 0 and thereafter as follows: 100 ul PBS (vehicle) subcutaneously every three days (Group 1); 0.1 mg/mouse IMM-101 subcutaneously adjacent to the tumour [peritumoral] every three days (Group 2); anti-PD1 intraperitoneally [RMP1-14] twice a week (Group 3), or a combination of anti-PD-1 [RMP1-14] twice a week and IMM-101 every 3 days (Group 4; IP and peritumoral, respectively). Mice were dosed until termination due to moribundity or a maximum tumour volume (TV) of 3000 mm³, whichever was the later.

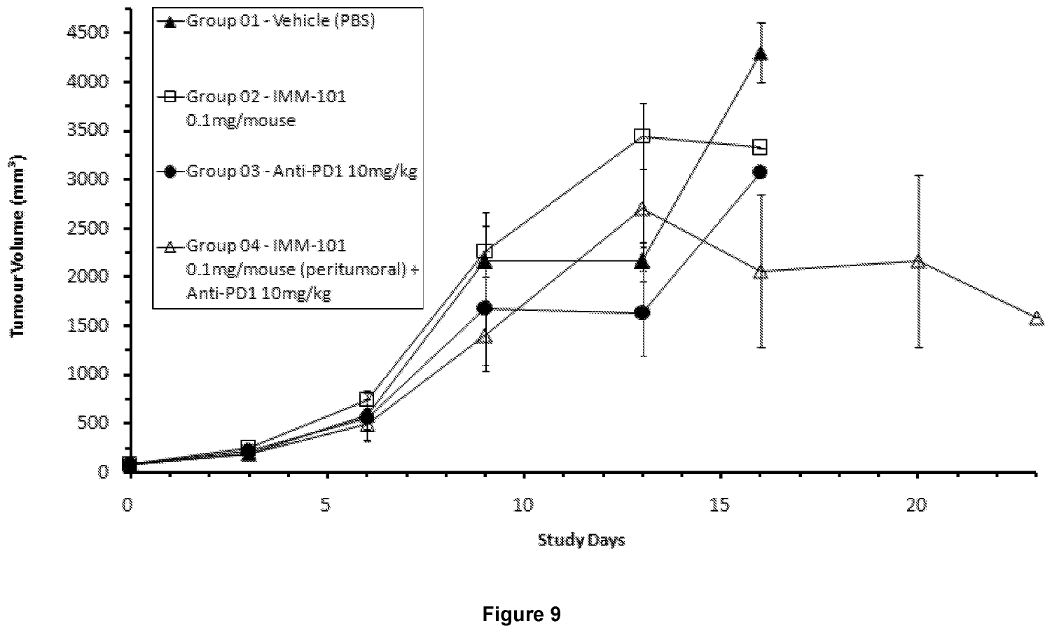

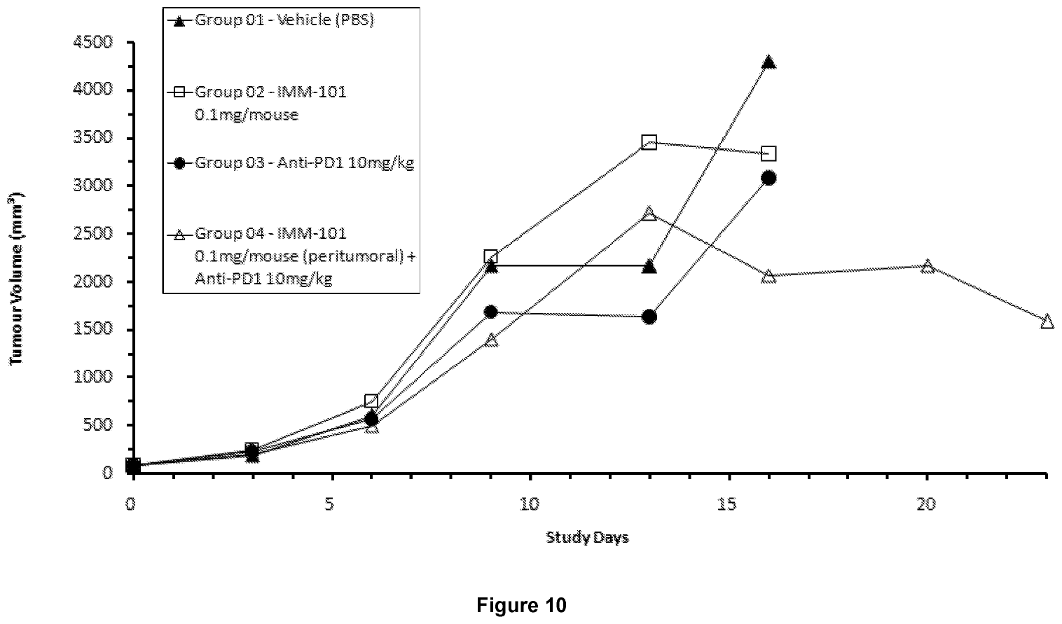

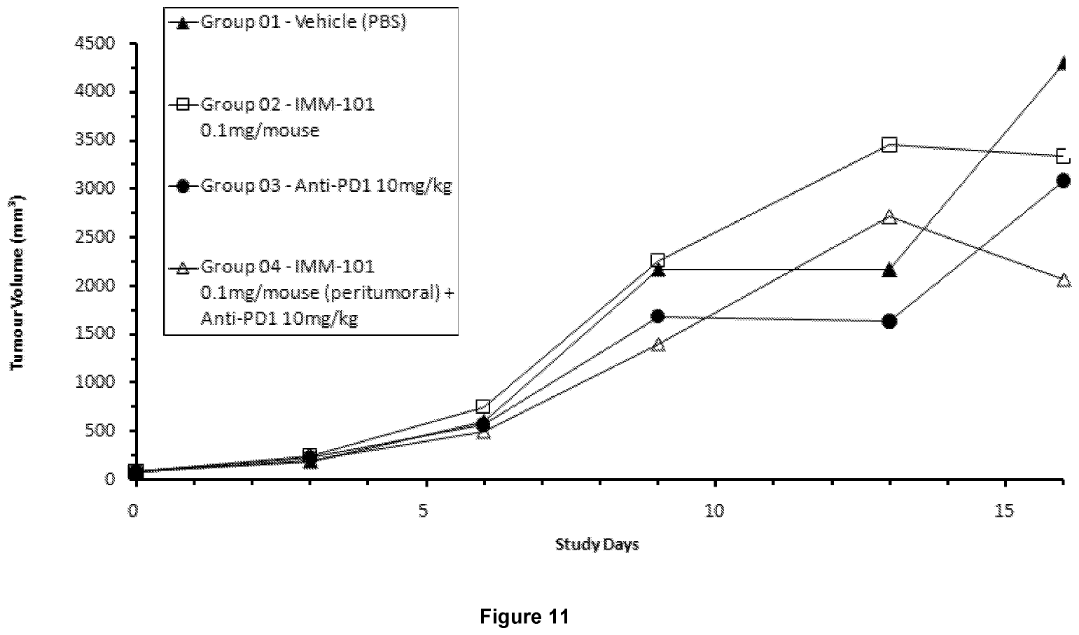

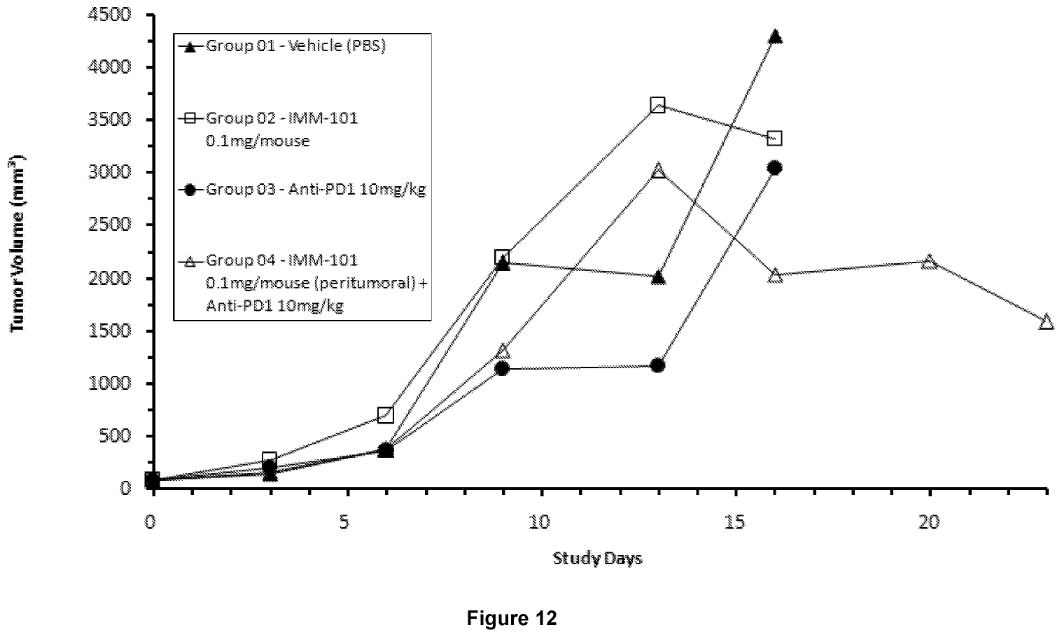

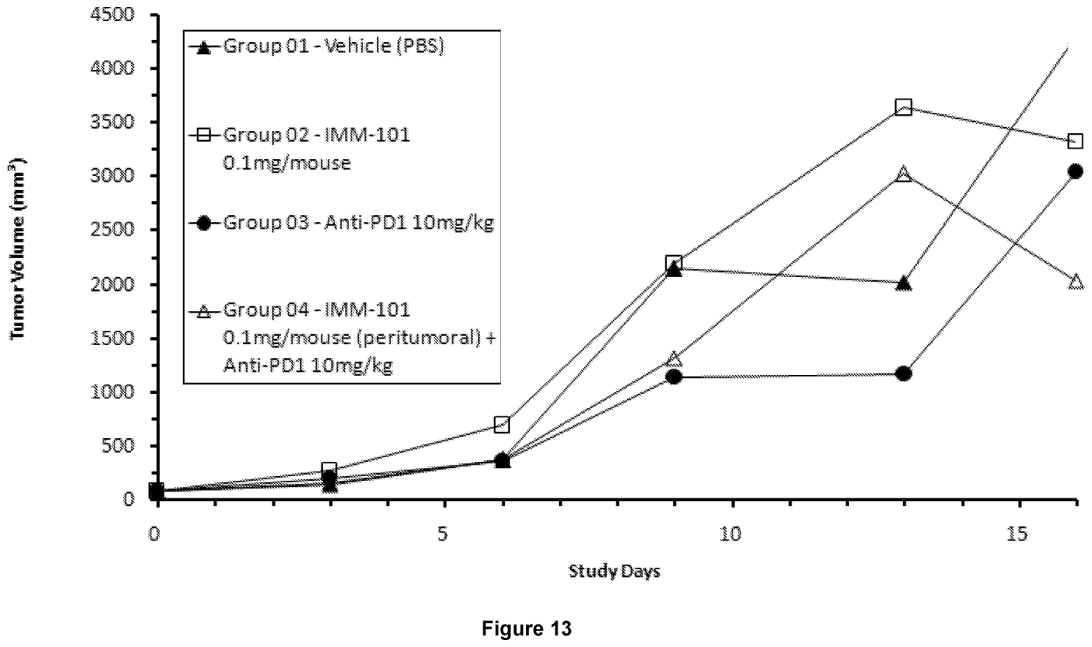

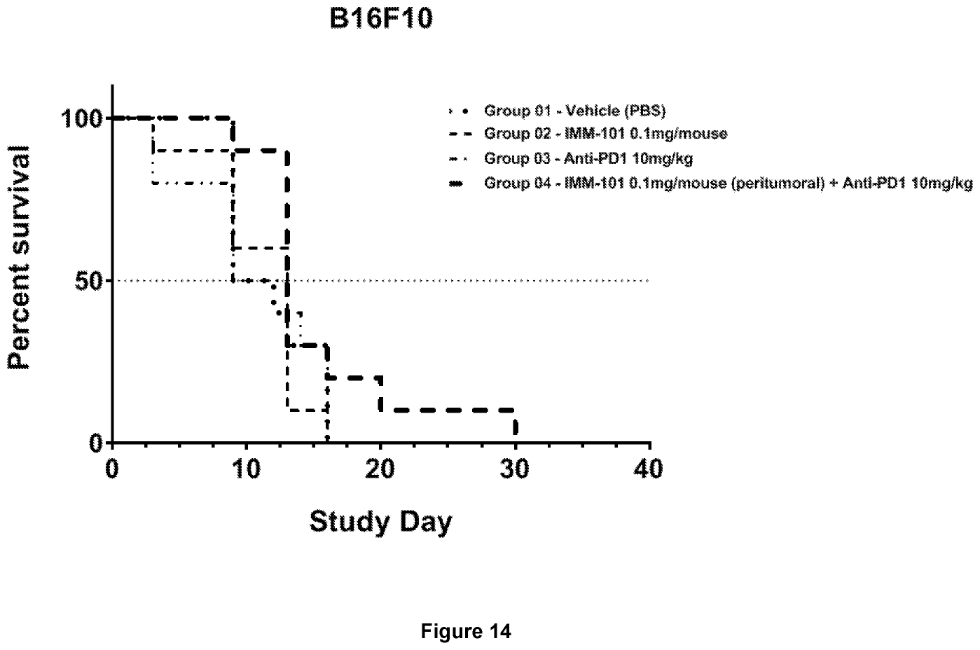

Results are presented in FIGS. 9 to 14, showing mean TV+/−SE (FIG. 9), mean TV without SE (FIG. 10), mean TV without SE up to study day 16 (FIG. 11), median TV (FIG. 12), median TV up to study day 16 (FIG. 13), and as a Kaplan-Meier survival graph (FIG. 14).

Calculations of tumour growth inhibition (%) for each group and study day indicated that Group 3 (anti-PD1 alone) had a % TGI of 29.07% at study day 16, whereas group 4 (IMM-101 plus anti-PD-1) demonstrated a % TGI of 52.90% at study day 16.

As can be seen from FIGS. 9 to 14 and in light of the % TGI values, the combination of IMM-101 and anti-PD1 has a marked increase in efficacy in this checkpoint refractory mouse model compared to anti-PD1 alone, particularly when the IMM-101 is administered subcutaneously adjacent to the tumour. Furthermore, said combination also results in a greater percentage survival compared to anti-PD1 alone.

### Example 7

The effects of combination treatment with IMM-101 (heat-killed whole cell M. obuense NTCT 13365) and a checkpoint inhibitor were investigated in C57BL/6j mice bearing subcutaneous checkpoint resistant Pan02 (pancreatic) tumours. The mice were inoculated with 3,000,000 tumour cells and then randomized with 10 mice per group when tumour volumes reached between 63 and 124 mm³ (mean TV ranged from 81 to 89 mm³ across the groups). Animals were dosed on Day 0 and thereafter as follows: 100 ul PBS (vehicle) subcutaneously every three days (Group 1); 0.1 mg/mouse IMM-101 subcutaneously adjacent to the tumour [peritumoral] every three days (Group 2); anti-PD1 intraperitoneally [RMP1-14] twice a week (Group 3), or a combination of anti-PD-1 [RMP1-14] twice a week and IMM-101 every 3 days (Group 4; IP and peritumoral, respectively). Mice were dosed until termination due to moribundity or a maximum tumour volume (TV) of 3000 mm³, whichever was the later.

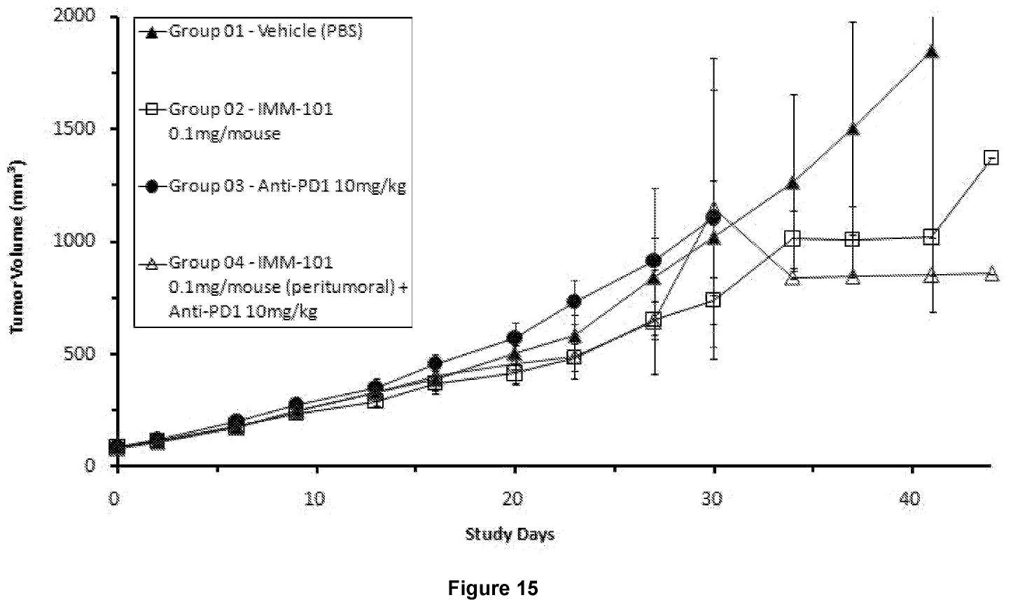

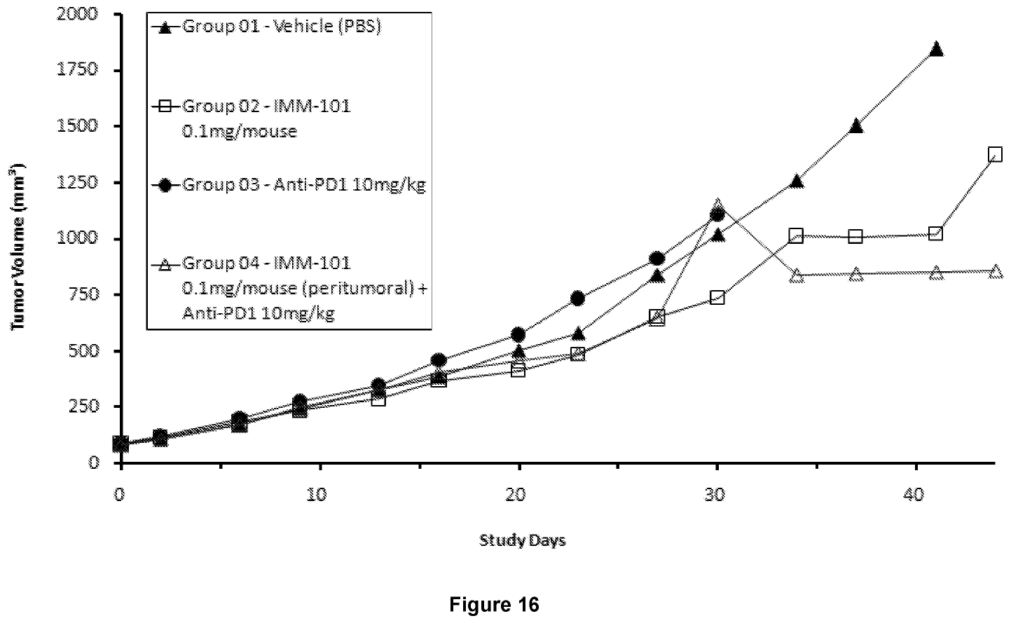

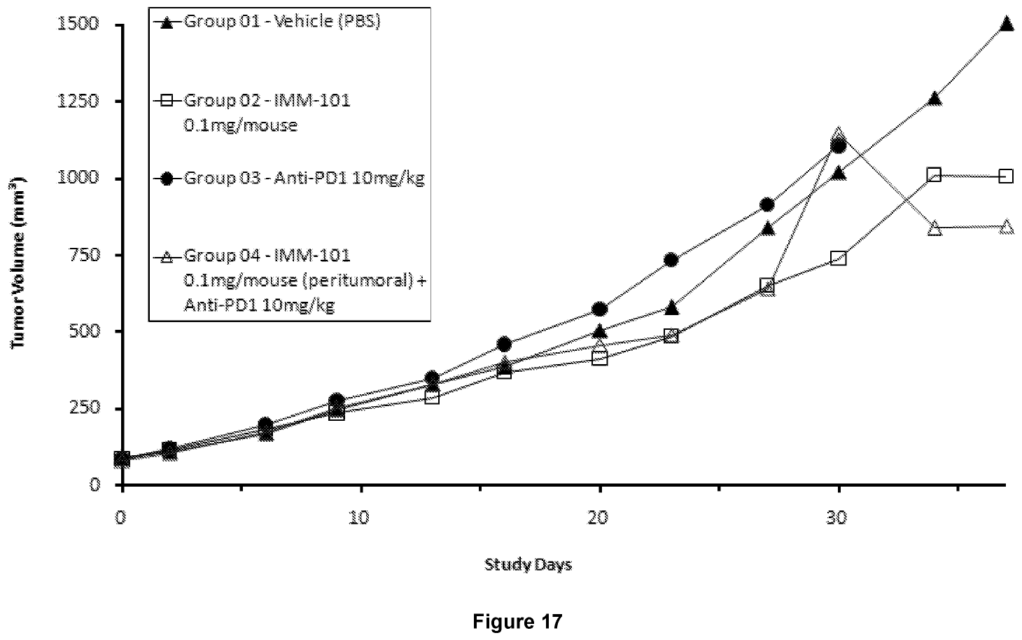

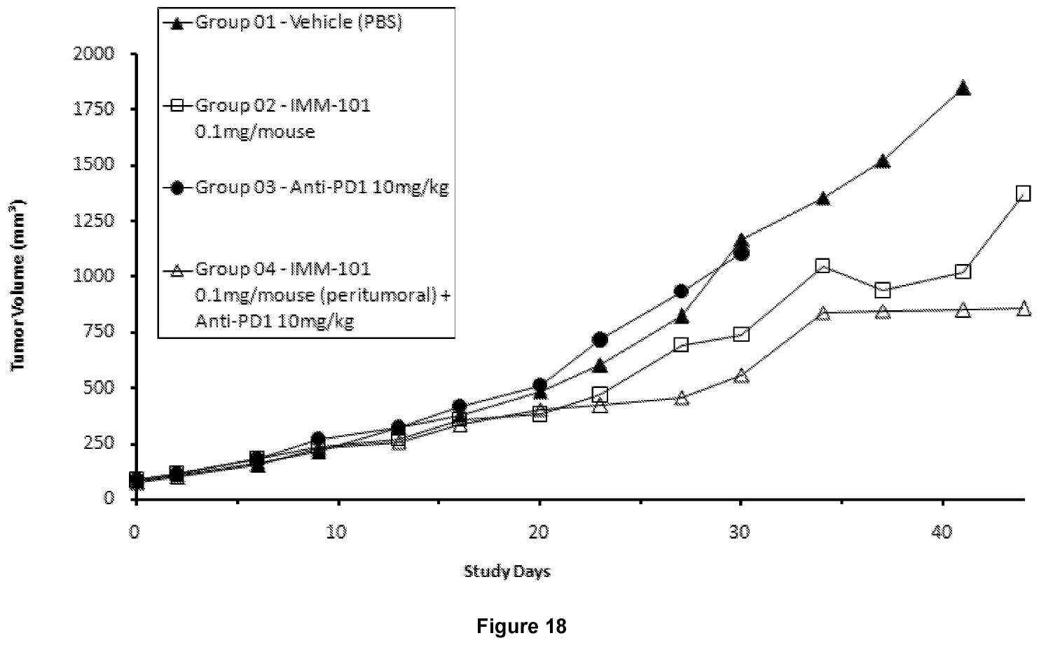

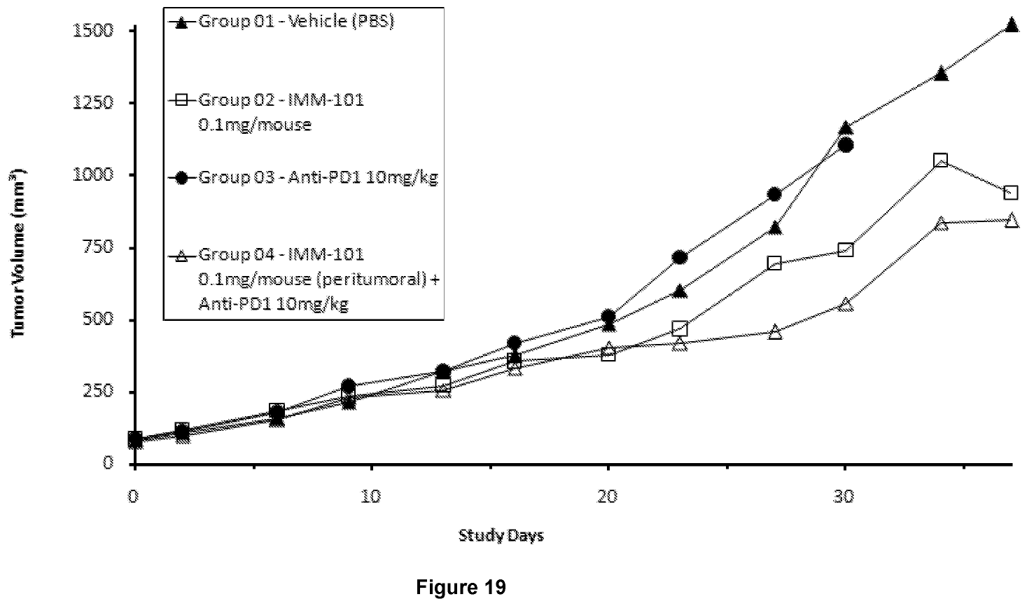

Results are presented in FIGS. 15 to 19, showing mean TV+/−SE (FIG. 15), mean TV without SE (FIG. 16), mean TV without SE up to study day 37 (FIG. 17), median TV (FIG. 18), and median TV up to study day 37 (FIG. 19).

Calculations of tumour growth inhibition (%) for each group and study day indicated that Group 3 (anti-PD1 alone) exhibited a maximum % TGI of −9.24% at study day 30 (when all mice in this group terminated), whereas group 4 (peritumoral IMM-101 plus anti-PD-1) demonstrated a % TGI of 56.22% at study day 41, and group 2 (peritumoral IMM-101 alone) exhibited a % TGI at study day 41 of 46.93%.

As can be seen from FIGS. 15 to 19 and in light of the % TGI values, the combination of IMM-101 and anti-PD1 has a marked increase in efficacy in this checkpoint refractory mouse model compared to anti-PD1 alone, particularly when the IMM-101 is administered subcutaneously adjacent to the tumour, alone or in combination with anti-D1. Furthermore, said combination or IMM-101 monotherapy also results in a greater survival compared to anti-PD1 alone.

We claim:

1-57. (canceled)

58. A method of treating, reducing, inhibiting or controlling a neoplasia, tumour or cancer in a checkpoint inhibitor refractory subject, wherein said method comprises simultaneously, separately, or sequentially administering to the subject, (i) one or more checkpoint inhibitors, and (ii) a non-viable, whole cell Mycobacterium, wherein said method results in enhanced therapeutic efficacy relative to administration of the one or more checkpoint inhibitors or non-viable whole cell Mycobacterium alone.

59. The method according to claim 58, wherein the one or more checkpoint inhibitors is selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3 and combinations thereof.

60. The method according to claim 58, wherein said one or more checkpoint inhibitors are selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CTLA-4, PD-1 or PD-L1, and combinations thereof.

61. The method according to claim 58, wherein said one or more checkpoint inhibitors are selected from ipilimumab, nivolumab, pembrolizumab, azetolizumab, durvalumab, tremelimumab, spartalizumab, avelumab, sintilimab, toripalimab, MGA012, MGD013, MGD019, enoblituzumab, MGD009, MGC018, MEDI0680, PDR001, FAZ053, TSR022, MBG453, relatlimab (BMS986016), LAG525, IMP321, REGN2810 (cemiplimab), REGN3767, pexidartinib, LY3022855, FPA008, BLZ945, GDC0919, epacadostat, indoximod, BMS986205, CPI-444, MEDI9447, PBF509, lirilumab and combinations thereof.

62. The method according to claim 58, wherein said one or more checkpoint inhibitors are selected from ipilimumab, nivolumab, or a combination selected from: durvalumab+tremelimumab, nivolumab+ipilimumab, pembrolizumab+ipilimumab, MEDI0680+durvalumab, PDR001+FAZ053, Nivolumab+TSR022, PDR001+MBG453, Nivolumab+BMS 986016 (relatlimab), PDR001+LAG 525, Pembrolizumab+IMP321, REGN2810 (cemiplimab)+REGN3767.

63. The method according to claim 58, wherein said checkpoint inhibition therapy further comprises co-stimulatory checkpoint therapy, simultaneously, separately or sequentially with administration of the Mycobacterium, wherein said co-stimulatory checkpoint therapy comprises administration of one or more checkpoint inhibitors, selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CD27, CD28, CD40, CD122, CD137, OX40, GITR, ICOS and combinations thereof, wherein said method results in enhanced therapeutic efficacy relative to administration of the one or more checkpoint inhibitors, co-stimulatory checkpoint therapy, or non-viable whole cell Mycobacterium alone.

64. The method according to claim 63, wherein said one or more checkpoint inhibitors are selected from utomilumab, urelumab, MOXR0916, PF04518600, MEDI0562, GSK3174988, MEDI6469, RO7009789, CP870893, BMS986156, GWN323, JTX-2011, varlilumab, MK-4166, NKTR-214 and combinations thereof.

65. The method according to claim 58, wherein administration of said non-viable whole-cell Mycobacterium is prior to and/or after the checkpoint inhibition therapy and/or the co-stimulatory checkpoint therapy.

66. The method according to claim 58, further comprising administering one or more additional anticancer treatments or agents, simultaneously, separately or sequentially with administration of the Mycobacterium, wherein said method results in enhanced therapeutic efficacy relative to administration of the one or more checkpoint inhibitors, co-stimulatory checkpoint therapy, one or more additional anticancer treatments or agents, or non-viable, whole cell Mycobacterium alone.

67. The method according to claim 66, wherein the one or more additional anticancer treatments or agents is selected from: adoptive cell therapy, surgical therapy, chemotherapy, radiation therapy, hormonal therapy, small molecule therapy such as metformin, receptor kinase inhibitor therapy, hyperthermia treatment, phototherapy, radioablation therapy, anti-angiogenic therapy, cytokine therapy, cryotherapy, biological therapy, HDAC inhibitor e.g. OKI-179, BRAF inhibitor, MEK inhibitor, EGFR inhibitor, VEGF inhibitor, P13K delta inhibitor, PARP inhibitor, mTOR inhibitor, hypomethylating agents, oncolytic virus, TLR agonist including TLR2, 3, 4, 5, 7, 8 or 9 agonists, such as MRx0518 (4D Pharma), STING agonists (including MIW815 and SYNB1891), and cancer vaccines such as GVAX or CIMAvax.

68. The method according to claim 67, wherein said TLR agonists include mifamurtide (Mepact), Krestin (PSK), MRx0518 (4D Pharma), IMO-2125 (tilsotolimod), CMP-001, MGN-1703 (lefitolimod), entolimod, SD-101, GS-9620, imiquimod, resiquimod, MEDI4736, poly I:C, CPG7909, DSP-0509, VTX-2337 (motolimod), MEDI9197, NKTR-262, G-100 or PF-3512676 and combinations thereof.

69. The method according to claim 67, wherein said chemotherapy comprises administration of one or more agents selected from: cyclophosphamide, methotrexate, 5-fluorouracil, doxorubicin, mustine, vincristine, procarbazine, prednisolone, bleomycin, vinblastine, dacarbazine, etoposide, cisplatin, epirubicin, capecitabine, leucovorin, folinic acid, carboplatin, oxaliplatin, gemcitabine, FOLFIRINOX, paclitaxel, pemetrexed, irinotecan and combinations thereof.

70. The method according to claim 67, wherein said one or more additional anticancer treatments or agents is administered intratumorally, intraarterially, intravenously, intravascularly, intrapleurally, intraperitoneally, intratracheally, intranasally, pulmonarily, intrathecally, intramuscularly, endoscopically, intralesionally, percutaneously, subcutaneously, regionally, stereotactically, orally or by direct injection or perfusion.

71. The method according to claim 58, wherein said neoplasia, tumour, or cancer is associated with a sarcoma, preferably a soft tissue or non-soft tissue sarcoma.

72. The method according to claim 58, wherein said neoplasia, tumour or cancer is associated with a cancer selected from prostate cancer, liver cancer, renal cancer, lung cancer, breast cancer, colorectal cancer, pancreatic cancer, brain cancer, hepatocellular cancer, lymphoma, leukaemia, gastric cancer, cervical cancer, ovarian cancer, thyroid cancer, head and neck cancer and skin cancer including melanoma.

73. The method according to claim 58, wherein said neoplasia, tumour or cancer is associated with pancreatic, colorectal, prostate, skin cancer, including melanoma or ovarian cancer.

74. The method according to claim 58, wherein the neoplasia, tumour, or cancer is metastatic.

75. The method according to claim 58, wherein the non-viable whole cell Mycobacterium is selected from M. vaccae, M. obuense, M. parafortuitum, M. aurum, M. indicus pranii, M. phlei and combinations thereof.

76. The method according to claim 58, wherein the non-viable whole cell Mycobacterium is the rough variant.

77. The method according to claim 58, wherein the non-viable whole cell Mycobacterium and/or checkpoint inhibitor is administered via the parenteral, oral, sublingual, nasal or pulmonary route.

78. The method according to claim 77, wherein the parenteral route is selected from subcutaneous, intradermal, subdermal, intraperitoneal, or intravenous.

79. The method according to claim 77, wherein the parenteral route comprises intratumoral, peritumoral, perilesional or intralesional administration.

80. The method according to claim 58, wherein the non-viable whole cell Mycobacterium is administered in an amount of from about 10⁴ to about 10¹⁰ cells, preferably about 10⁷ to about 10⁹ cells.

81. The method according to claim 58, wherein enhanced therapeutic efficacy is measured by increased overall survival time.

82. The method according to claim 58, wherein enhanced therapeutic efficacy is measured by increased progression-free survival.

83. The method according to claim 58, wherein said enhanced therapeutic efficacy is measured by a decrease or stabilisation of tumour size of one or more said tumours, as defined by RECIST 1.1, including stable diseases (SD), a complete response (CR) or partial response (PR) of the target tumour; and/or stable disease (SD) or complete response (CR) of one or more non-target tumours.

84. The method according to claim 58, wherein enhanced therapeutic efficacy is measured by an improved overall response rate and/or increased quality of life.

85. The method according to claim 58, wherein said checkpoint inhibitor refractory patient exhibits an innate (primary) resistance to checkpoint inhibitor therapy as demonstrated by a lack of response or an insufficient response to said checkpoint inhibitor therapy, for at least about 8 weeks, or 12 weeks, or an acquired (secondary) resistance to checkpoint inhibitor therapy as demonstrated by an initial response to said checkpoint therapy but with a subsequent relapse and progression of one or more tumours.

86. The method according to claim 58, wherein said checkpoint inhibitor refractory patient exhibits an innate (primary) resistance or an acquired (secondary) resistance to treatment with one or more CTLA-4, PD-1, PD-L1, PD-L2, B7-H3, B7-H4, B7-H6, A2AR, IDO, TIM-3, BTLA, VISTA, LAG-3 inhibitors.

87. The method according to claim 58, wherein said administering of (ii) comprises:
providing a microneedle device comprising a plurality of microneedles;
causing the microneedles to penetrate into the skin of the subject and assume an anchored state in which the microneedles are anchored in the skin and project from the microneedle device; and
delivering into the skin via the microneedle a quantity of the whole cell, non-viable Mycobacterium.

88. A kit of parts for delivering a non-viable whole-cell Mycobacterium into the skin of a checkpoint inhibitor refractory patient, comprising:
a microneedle device comprising a plurality of microneedles; and
a whole cell non-viable Mycobacterium such as M. vaccae, such as NCTC 11569, M. obuense, such as NCTC 13365, M. parafortuitum, M. aurum, M. indicus pranii, M. phlei, and combinations thereof; and
a checkpoint inhibitor selected from a cell, protein, peptide, antibody, or antigen binding fragment thereof, directed against CTLA-4, PD-1, PD-L1, PD-L2, LAG-3, B7-H3, B7-H4, B7-H6, A2AR, or IDO, and combinations thereof.
